SEQUENCE LISTING (259 sequences)

SEQ ID NO: 1 - RNA, 20 nt, Artificial Sequence
  gRNA of B2M-01
gaguagcgcgagcacagcua

SEQ ID NO: 2 - RNA, 20 nt, Artificial Sequence
  gRNA of B2M-03
cucgcgcuacucucucuuuc

SEQ ID NO: 3 - RNA, 20 nt, Artificial Sequence
  gRNA of B2M-04
gcauacucaucuuuuucagu

SEQ ID NO: 4 - RNA, 20 nt, Artificial Sequence
  gRNA of B2M-05
gcuacucucucuuucuggcc

SEQ ID NO: 5 - RNA, 20 nt, Artificial Sequence
  gRNA of B2M-06
ggcauacucaucuuuuucag

SEQ ID NO: 6 - RNA, 20 nt, Artificial Sequence
  gRNA of B2M-07
ggccacggagcgagacaucu

SEQ ID NO: 7 - RNA, 20 nt, Artificial Sequence
  gRNA of B2M-08
ggccgagaugucucgcuccg

SEQ ID NO: 8 - RNA, 20 nt, Artificial Sequence
  gRNA of B2M-09
ucacgucauccagcagagaa

SEQ ID NO: 9 - RNA, 20 nt, Artificial Sequence
  gRNA of B2M-10
acaaagucacaugguucaca

SEQ ID NO: 10 - RNA, 20 nt, Artificial Sequence
  gRNA of B2M-11
agucacaugguucacacggc

SEQ ID NO: 11 - RNA, 20 nt, Artificial Sequence
  gRNA of B2M-12
aagucaacuucaaugucgga

SEQ ID NO: 12 - RNA, 20 nt, Artificial Sequence
  gRNA of B2M-13
cauacucaucuuuuucagug

SEQ ID NO: 13 - RNA, 20 nt, Artificial Sequence
  gRNA of B2M-14
uccugaauugcuaugugucu

SEQ ID NO: 14 - RNA, 20 nt, Artificial Sequence
  gRNA of B2M-15
cgugaguaaaccugaaucuu

SEQ ID NO: 15 - RNA, 20 nt, Artificial Sequence
  gRNA of B2M-16
uuggaguaccugaggaauau

SEQ ID NO: 16 - RNA, 20 nt, Artificial Sequence
  gRNA of B2M-17
aggguaggagagacucacgc

SEQ ID NO: 17 - RNA, 20 nt, Artificial Sequence
  gRNA of B2M-18
acagcccaagauaguuaagu

SEQ ID NO: 18 - RNA, 20 nt, Artificial Sequence
  gRNA of B2M-19
auacucaucuuuuucagugg

SEQ ID NO: 19 - RNA, 20 nt, Artificial Sequence
  gRNA of B2M-20
uggaguaccugaggaauauc

SEQ ID NO: 20 - RNA, 20 nt, Artificial Sequence
  gRNA of B2M-21
aagaaaaggaaacugaaaac

SEQ ID NO: 21 - RNA, 20 nt, Artificial Sequence
  gRNA of B2M-22
aagaaggcaugcacuagacu

SEQ ID NO: 22 - RNA, 20 nt, Artificial Sequence
  gRNA of B2M-23
acauguaagcagcaucaugg

SEQ ID NO: 23 - RNA, 20 nt, Artificial Sequence
  gRNA of B2M-24
acccagacacauagcaauuc

SEQ ID NO: 24 - RNA, 20 nt, Artificial Sequence
  gRNA of B2M-25
acuugucuuucagcaaggac

SEQ ID NO: 25 - RNA, 20 nt, Artificial Sequence
  gRNA of B2M-26
caagccagcgacgcagugcc

SEQ ID NO: 26 - RNA, 20 nt, Artificial Sequence
  gRNA of B2M-27
cacagcccaagauaguuaag

SEQ ID NO: 27 - RNA, 20 nt, Artificial Sequence
  gRNA of B2M-29
caucacgagacucuaagaaa

SEQ ID NO: 28 - RNA, 20 nt, Artificial Sequence
  gRNA of B2M-30
cgcagugccagguuagagag

SEQ ID NO: 29 - RNA, 20 nt, Artificial Sequence
  gRNA of B2M-31
cuaaccuggcacugcgucgc

SEQ ID NO: 30 - RNA, 20 nt, Artificial Sequence
  gRNA of B2M-32
gaaagucccucucucuaacc

SEQ ID NO: 31 - RNA, 20 nt, Artificial Sequence
  gRNA of B2M-33
gagacauguaagcagcauca

SEQ ID NO: 32 - RNA, 20 nt, Artificial Sequence
  gRNA of B2M-34
gagucucgugauguuuaaga

SEQ ID NO: 33 - RNA, 20 nt, Artificial Sequence
  gRNA of B2M-35
gcagugccagguuagagaga

SEQ ID NO: 34 - RNA, 20 nt, Artificial Sequence
  gRNA of B2M-36
uaagaaggcaugcacuagac

SEQ ID NO: 35 - RNA, 20 nt, Artificial Sequence
  gRNA of B2M-37
ucgaucuaugaaaaagacag

SEQ ID NO: 36 - RNA, 20 nt, Artificial Sequence
  gRNA of B2M-39
uucagacuugucuuucagca

SEQ ID NO: 37 - RNA, 20 nt, Artificial Sequence
  gRNA of B2M-40
uuccugaauugcuauguguc

SEQ ID NO: 38 - RNA, 20 nt, Artificial Sequence
  gRNA of B2M-41
uaagaaaaggaaacugaaaa

SEQ ID NO: 39 - RNA, 20 nt, Artificial Sequence
  gRNA of B2M-42
cuggcacugcgucgcuggcu

SEQ ID NO: 40 - RNA, 20 nt, Artificial Sequence
  gRNA of B2M-43
ugcgucgcuggcuuggagac

SEQ ID NO: 41 - RNA, 20 nt, Artificial Sequence
  gRNA of B2M-44
gcuggcuuggagacagguga

SEQ ID NO: 42 - RNA, 20 nt, Artificial Sequence
  gRNA of B2M-45
agacaggugacggucccugc

SEQ ID NO: 43 - RNA, 20 nt, Artificial Sequence
  gRNA of B2M-46
caaucaggacaaggcccgca

SEQ ID NO: 44 - RNA, 20 nt, Artificial Sequence
  gRNA of B2M-47
ccugcgggccuuguccugau

SEQ ID NO: 45 - RNA, 20 nt, Artificial Sequence
  gRNA of B2M-48
ccaaucaggacaaggcccgc

SEQ ID NO: 46 - RNA, 20 nt, Artificial Sequence
  gRNA of B2M-49
cgggccuuguccugauuggc

SEQ ID NO: 47 - RNA, 20 nt, Artificial Sequence
  gRNA of B2M-50
gggccuuguccugauuggcu

SEQ ID NO: 48 - RNA, 20 nt, Artificial Sequence
  gRNA of B2M-51
gugcccagccaaucaggaca

SEQ ID NO: 49 - RNA, 20 nt, Artificial Sequence
  gRNA of B2M-52
aaacgcgugcccagccaauc

SEQ ID NO: 50 - RNA, 20 nt, Artificial Sequence
  gRNA of B2M-53
gggcacgcguuuaauauaag

SEQ ID NO: 51 - RNA, 20 nt, Artificial Sequence
  gRNA of B2M-54
cacgcguuuaauauaagugg

SEQ ID NO: 52 - RNA, 20 nt, Artificial Sequence
  gRNA of B2M-55
uauaaguggaggcgucgcgc

SEQ ID NO: 53 - RNA, 20 nt, Artificial Sequence
  gRNA of B2M-56
aaguggaggcgucgcgcugg

SEQ ID NO: 54 - RNA, 20 nt, Artificial Sequence
  gRNA of B2M-57
aguggaggcgucgcgcuggc

SEQ ID NO: 55 - RNA, 20 nt, Artificial Sequence
  gRNA of B2M-58
uuccugaagcugacagcauu

SEQ ID NO: 56 - RNA, 20 nt, Artificial Sequence
  gRNA of B2M-59
uccugaagcugacagcauuc

SEQ ID NO: 57 - RNA, 20 nt, Artificial Sequence
  gRNA of B2M-60
ugggcugugacaaagucaca

SEQ ID NO: 58 - RNA, 20 nt, Artificial Sequence
  gRNA of B2M-61
acucucucuuucuggccugg

SEQ ID NO: 59 - RNA, 20 nt, Artificial Sequence
  gRNA of DQA-08
uuaggaucauccucuuccca

SEQ ID NO: 60 - RNA, 20 nt, Artificial Sequence
  gRNA of DQA-09
aacucuaccgcugcuaccaa

SEQ ID NO: 61 - RNA, 20 nt, Artificial Sequence
  gRNA of DQA-10
acaaugucuucaccuccaca

SEQ ID NO: 62 - RNA, 20 nt, Artificial Sequence
  gRNA of DQA-11
accaccgugaugagccccug

SEQ ID NO: 63 - RNA, 20 nt, Artificial Sequence
  gRNA of DQA-12
acccagugucacgggagacu

SEQ ID NO: 64 - RNA, 20 nt, Artificial Sequence
  gRNA of DQA-14
accuccacaggggcucauca

SEQ ID NO: 65 - RNA, 20 nt, Artificial Sequence
  gRNA of DQA-15
caaugucuucaccuccacag

SEQ ID NO: 66 - RNA, 20 nt, Artificial Sequence
  gRNA of DQA-16
cacaaugucuucaccuccac

SEQ ID NO: 67 - RNA, 20 nt, Artificial Sequence
  gRNA of DQA-17
caguacacccaugaauuuga

SEQ ID NO: 68 - RNA, 20 nt, Artificial Sequence
  gRNA of DQA-18
cucugugagcucugacauag

SEQ ID NO: 69 - RNA, 20 nt, Artificial Sequence
  gRNA of DQA-19
cuguggaggugaagacauug

SEQ ID NO: 70 - RNA, 20 nt, Artificial Sequence
  gRNA of DQA-20
ggcuggaaucucaggcucug

SEQ ID NO: 71 - RNA, 20 nt, Artificial Sequence
  gRNA of DQA-21
guugggcugacccaguguca

SEQ ID NO: 72 - RNA, 20 nt, Artificial Sequence
  gRNA of DQA-22
ucauggguguacuggccaga

SEQ ID NO: 73 - RNA, 20 nt, Artificial Sequence
  gRNA of DQA-23
uccaagucucccgugacacu

SEQ ID NO: 74 - RNA, 20 nt, Artificial Sequence
  gRNA of DQA-24
uccacaggggcucaucacgg

SEQ ID NO: 75 - RNA, 20 nt, Artificial Sequence
  gRNA of DQA-25
uguggaggugaagacauugu

SEQ ID NO: 76 - RNA, 20 nt, Artificial Sequence
  gRNA of DQA-26
uuccaagucucccgugacac

SEQ ID NO: 77 - RNA, 20 nt, Artificial Sequence
  gRNA of DQA-27
uugggcugacccagugucac

SEQ ID NO: 78 - RNA, 20 nt, Artificial Sequence
  gRNA of DQA-28
aacaucacauggcugagcaa

SEQ ID NO: 79 - RNA, 20 nt, Artificial Sequence
  gRNA of DQA-29
acaucacauggcugagcaau

SEQ ID NO: 80 - RNA, 20 nt, Artificial Sequence
  gRNA of DQA-30
agccaugugauguugaccac

SEQ ID NO: 81 - RNA, 20 nt, Artificial Sequence
  gRNA of DQA-31
aggaaugaucacucuuggag

SEQ ID NO: 82 - RNA, 20 nt, Artificial Sequence
  gRNA of DQA-32
aucacucuuggagaggaagc

SEQ ID NO: 83 - RNA, 20 nt, Artificial Sequence
  gRNA of DQA-33
augacugcaagguggagcac

SEQ ID NO: 84 - RNA, 20 nt, Artificial Sequence
  gRNA of DQA-34
caagguggagcacuggggcc

SEQ ID NO: 85 - RNA, 20 nt, Artificial Sequence
  gRNA of DQA-35
caucaaauucauggguguac

SEQ ID NO: 86 - RNA, 20 nt, Artificial Sequence
  gRNA of DQA-36
caugugauguugaccacagg

SEQ ID NO: 87 - RNA, 20 nt, Artificial Sequence
  gRNA of DQA-37
ccucaccacagagguuccug

SEQ ID NO: 88 - RNA, 20 nt, Artificial Sequence
  gRNA of DQA-38
cucaucuccaucaaauucau

SEQ ID NO: 89 - RNA, 20 nt, Artificial Sequence
  gRNA of DQA-39
cuccuguggucaacaucaca

SEQ ID NO: 90 - RNA, 20 nt, Artificial Sequence
  gRNA of DQA-40
gaagaaggaaugaucacucu

SEQ ID NO: 91 - RNA, 20 nt, Artificial Sequence
  gRNA of DQA-41
gacugcaagguggagcacug

SEQ ID NO: 92 - RNA, 20 nt, Artificial Sequence
  gRNA of DQA-42
gagguaacugaucuugaaga

SEQ ID NO: 93 - RNA, 20 nt, Artificial Sequence
  gRNA of DQA-43
ggacaacaucuuuccuccug

SEQ ID NO: 94 - RNA, 20 nt, Artificial Sequence
  gRNA of DQA-44
gugcuguuuccucaccacag

SEQ ID NO: 95 - RNA, 20 nt, Artificial Sequence
  gRNA of DQA-45
ucuucugaaacacuggggua

SEQ ID NO: 96 - RNA, 20 nt, Artificial Sequence
  gRNA of DQA-47
uucauggguguacuggccag

SEQ ID NO: 97 - RNA, 20 nt, Artificial Sequence
  gRNA of DQA-48
agagacuguggucugcgccc

SEQ ID NO: 98 - RNA, 20 nt, Artificial Sequence
  gRNA of DQA-49
gacauaggggcuggaaucuc

SEQ ID NO: 99 - RNA, 20 nt, Artificial Sequence
  gRNA of DQA-50
gagacuguggucugcgcccu

SEQ ID NO: 100 - RNA, 20 nt, Artificial Sequence
  gRNA of DQA-51
ggccucgugggcauuguggu

SEQ ID NO: 101 - RNA, 20 nt, Artificial Sequence
  gRNA of DQA-52
gggccucgugggcauugugg

SEQ ID NO: 102 - RNA, 20 nt, Artificial Sequence
  gRNA of DQA-53
gucagagcucacagagacug

SEQ ID NO: 103 - RNA, 20 nt, Artificial Sequence
  gRNA of DQA-54
gucucugugagcucugacau

SEQ ID NO: 104 - RNA, 20 nt, Artificial Sequence
  gRNA of DQA-55
gugagcucugacauaggggc

SEQ ID NO: 105 - RNA, 20 nt, Artificial Sequence
  gRNA of DQA-56
guuggugcuuccagacacca

SEQ ID NO: 106 - RNA, 20 nt, Artificial Sequence
  gRNA of DQA-57
ucucugugagcucugacaua

SEQ ID NO: 107 - RNA, 20 nt, Artificial Sequence
  gRNA of DQA-58
ugacugcaagguggagcacu

SEQ ID NO: 108 - RNA, 20 nt, Artificial Sequence
  gRNA of DQA-59
ugcccaccacaaugcccacg

SEQ ID NO: 109 - RNA, 20 nt, Artificial Sequence
  gRNA of DQA-60
uggaagcaccaacugaacgc

SEQ ID NO: 110 - RNA, 20 nt, Artificial Sequence
  gRNA of DQA-61
ugugggccucgugggcauug

SEQ ID NO: 111 - RNA, 20 nt, Artificial Sequence
  gRNA of DQA-62
uuaccccaguguuucagaag

SEQ ID NO: 112 - RNA, 20 nt, Artificial Sequence
  gRNA of DQA-63
uuggaaaacacugugaccuc

SEQ ID NO: 113 - RNA, 20 nt, Artificial Sequence
  gRNA of DQA-64
uuggugcuuccagacaccaa

SEQ ID NO: 114 - RNA, 20 nt, Artificial Sequence
  gRNA of DQA-65
aaacaaagcucugcugcugg

SEQ ID NO: 115 - RNA, 20 nt, Artificial Sequence
  gRNA of DQA-66
aaaucucaucagcagaaggg

SEQ ID NO: 116 - RNA, 20 nt, Artificial Sequence
  gRNA of DQA-67
cuaaacaaagcucugcugcu

SEQ ID NO: 117 - RNA, 20 nt, Artificial Sequence
  gRNA of DPA-01
ucuaugcgucuguacaaacg

SEQ ID NO: 118 - RNA, 20 nt, Artificial Sequence
  gRNA of DPA-02
guacagacgcauagaccaac

SEQ ID NO: 119 - RNA, 20 nt, Artificial Sequence
  gRNA of DPA-03
uacagacgcauagaccaaca

SEQ ID NO: 120 - RNA, 20 nt, Artificial Sequence
  gRNA of DPA-04
gaaggagaccgucuggcauc

SEQ ID NO: 121 - RNA, 20 nt, Artificial Sequence
  gRNA of DPA-05
ggagaccgucuggcaucugg

SEQ ID NO: 122 - RNA, 20 nt, Artificial Sequence
  gRNA of DPA-06
gucuggcaucuggaggaguu

SEQ ID NO: 123 - RNA, 20 nt, Artificial Sequence
  gRNA of DPA-07
gugguuggaacgcuggauca

SEQ ID NO: 124 - RNA, 20 nt, Artificial Sequence
  gRNA of DPA-08
gucuucagggcgcauguugu

SEQ ID NO: 125 - RNA, 20 nt, Artificial Sequence
  gRNA of DPA-09
ugucuucagggcgcauguug

SEQ ID NO: 126 - RNA, 20 nt, Artificial Sequence
  gRNA of DPA-10
ucuucagggcgcauguugug

SEQ ID NO: 127 - RNA, 20 nt, Artificial Sequence
  gRNA of DPA-11
guugcauaccccagugcuug

SEQ ID NO: 128 - RNA, 20 nt, Artificial Sequence
  gRNA of DPA-12
gaccuuugugcccucagcag

SEQ ID NO: 129 - RNA, 20 nt, Artificial Sequence
  gRNA of DPA-13
gagacucagcaggaaagcca

SEQ ID NO: 130 - RNA, 20 nt, Artificial Sequence
  gRNA of DPA-14
gagccucaaaggaaaaggcu

SEQ ID NO: 131 - RNA, 20 nt, Artificial Sequence
  gRNA of DPA-15
gaucuugagagcccucuccu

SEQ ID NO: 132 - RNA, 20 nt, Artificial Sequence
  gRNA of DPA-16
gccaucaagggugagugcuc

SEQ ID NO: 133 - RNA, 20 nt, Artificial Sequence
  gRNA of DPA-17
gccaugacccccgggcccag

SEQ ID NO: 134 - RNA, 20 nt, Artificial Sequence
  gRNA of DPA-18
gcccagcuccacaggcuccu

SEQ ID NO: 135 - RNA, 20 nt, Artificial Sequence
  gRNA of DPA-19
gcccugagccucaaaggaaa

SEQ ID NO: 136 - RNA, 20 nt, Artificial Sequence
  gRNA of DPA-20
gccuuuuccuuugaggcuca

SEQ ID NO: 137 - RNA, 20 nt, Artificial Sequence
  gRNA of DPA-21
gcguucuggccaugaccccc

SEQ ID NO: 138 - RNA, 20 nt, Artificial Sequence
  gRNA of DPA-22
gcuuuccugcugagucuccg

SEQ ID NO: 139 - RNA, 20 nt, Artificial Sequence
  gRNA of DPA-23
ggaaacacggucaccucagg

SEQ ID NO: 140 - RNA, 20 nt, Artificial Sequence
  gRNA of DPA-24
ggacuucuaugacugcaggg

SEQ ID NO: 141 - RNA, 20 nt, Artificial Sequence
  gRNA of DPA-25
ggagacugugcucugugccc

SEQ ID NO: 142 - RNA, 20 nt, Artificial Sequence
  gRNA of DPA-26
ggccaugacccccgggccca

SEQ ID NO: 143 - RNA, 20 nt, Artificial Sequence
  gRNA of DPA-27
ggccuagucggcaucaucgu

SEQ ID NO: 144 - RNA, 20 nt, Artificial Sequence
  gRNA of DPA-29
gggaaacacggucaccucag

SEQ ID NO: 145 - RNA, 20 nt, Artificial Sequence
  gRNA of DPA-30
gggccuagucggcaucaucg

SEQ ID NO: 146 - RNA, 20 nt, Artificial Sequence
  gRNA of DPA-31
gucauagaaguccucugcug

SEQ ID NO: 147 - RNA, 20 nt, Artificial Sequence
  gRNA of DPA-32
guccucugcugagggcacaa

SEQ ID NO: 148 - RNA, 20 nt, Artificial Sequence
  gRNA of DPA-33
guggaagcuguaaucuguuc

SEQ ID NO: 149 - RNA, 20 nt, Artificial Sequence
  gRNA of DPA-34
gugggaagaacuugucaaug

SEQ ID NO: 150 - RNA, 20 nt, Artificial Sequence
  gRNA of DPA-35
guugguggccugaguguggu

SEQ ID NO: 151 - RNA, 20 nt, Artificial Sequence
  gRNA of DPA-36
guugucucaggcaucuggau

SEQ ID NO: 152 - RNA, 20 nt, Artificial Sequence
  gRNA of DPA-37
gcugagucuccgaggagcug

SEQ ID NO: 153 - RNA, 20 nt, Artificial Sequence
  gRNA of DPA-38
ucucuacugucuuuaugcag

SEQ ID NO: 154 - RNA, 20 nt, Artificial Sequence
  gRNA of DPA-39
uauggaacauucugucuuca

SEQ ID NO: 155 - RNA, 20 nt, Artificial Sequence
  gRNA of DPA-40
ucaagaucacagcucugaua

SEQ ID NO: 156 - RNA, 20 nt, Artificial Sequence
  gRNA of DPA-41
ucaaacauaaacuccccugu

SEQ ID NO: 157 - RNA, 20 nt, Artificial Sequence
  gRNA of DPA-42
uaccguugguggccugagug

SEQ ID NO: 158 - RNA, 20 nt, Artificial Sequence
  gRNA of DPA-43
uccugagcacucacccuuga

SEQ ID NO: 159 - RNA, 20 nt, Artificial Sequence
  gRNA of DPA-44
ugaggugaccguguuuccca

SEQ ID NO: 160 - RNA, 20 nt, Artificial Sequence
  gRNA of DPA-45
ugcguucuggccaugacccc

SEQ ID NO: 161 - RNA, 20 nt, Artificial Sequence
  gRNA of DPA-46
uuuccuuugaggcucagggc

SEQ ID NO: 162 - RNA, 20 nt, Artificial Sequence
  gRNA of DPA-47
ugccgacuaggcccagcacc

SEQ ID NO: 163 - RNA, 20 nt, Artificial Sequence
  gRNA of DPA-48
ucagcaggaaagccaaggag

SEQ ID NO: 164 - RNA, 20 nt, Artificial Sequence
  gRNA of DPA-49
ugaagaugagauguucuaug

SEQ ID NO: 165 - RNA, 20 nt, Artificial Sequence
  gRNA of DPA-50
ugcugagucuccgaggagcu

SEQ ID NO: 166 - RNA, 20 nt, Artificial Sequence
  gRNA of DPA-51
ugagauguucuauguggauc

SEQ ID NO: 167 - RNA, 20 nt, Artificial Sequence
  gRNA of DPA-52
ugggaaacacggucaccuca

SEQ ID NO: 168 - RNA, 20 nt, Artificial Sequence
  gRNA of DPA-53
uggaagcuguaaucuguucu

SEQ ID NO: 169 - RNA, 20 nt, Artificial Sequence
  gRNA of DPA-54
uggacaagaaggagaccguc

SEQ ID NO: 170 - RNA, 20 nt, Artificial Sequence
  gRNA of DPA-55
ugcccacgaugaugccgacu

SEQ ID NO: 171 - RNA, 20 nt, Artificial Sequence
  gRNA of DPA-56
uggccaagccuuuuccuuug

SEQ ID NO: 172 - RNA, 20 nt, Artificial Sequence
  gRNA of DPA-57
guggcugugcaacggggagc

SEQ ID NO: 173 - RNA, 20 nt, Artificial Sequence
  gRNA of DPA-58
uccccugggcccggggguca

SEQ ID NO: 174 - RNA, 20 nt, Artificial Sequence
  gRNA of DPA-59
ucaccucagggggaucugga

SEQ ID NO: 175 - RNA, 20 nt, Artificial Sequence
  gRNA of DPA-60
ucuccuuccagaucccccug

SEQ ID NO: 176 - RNA, 20 nt, Artificial Sequence
  gRNA of DRA-08
aagaagaaaauggccauaag

SEQ ID NO: 177 - RNA, 20 nt, Artificial Sequence
  gRNA of DRA-09
aaucaugggcuaucaaaggu

SEQ ID NO: 178 - RNA, 20 nt, Artificial Sequence
  gRNA of DRA-10
agcugugcugaugagcgcuc

SEQ ID NO: 179 - RNA, 20 nt, Artificial Sequence
  gRNA of DRA-11
auaaguggagucccugugcu

SEQ ID NO: 180 - RNA, 20 nt, Artificial Sequence
  gRNA of DRA-12
acuuauggccauuuucuucu

SEQ ID NO: 181 - RNA, 20 nt, Artificial Sequence
  gRNA of DRA-13
augaugaaaaauccuagcac

SEQ ID NO: 182 - RNA, 20 nt, Artificial Sequence
  gRNA of DRA-14
cagagcgcccaagaagaaaa

SEQ ID NO: 183 - RNA, 20 nt, Artificial Sequence
  gRNA of DRA-15
caggaaucaugggcuaucaa

SEQ ID NO: 184 - RNA, 20 nt, Artificial Sequence
  gRNA of DRA-16
cuuauggccauuuucuucuu

SEQ ID NO: 185 - RNA, 20 nt, Artificial Sequence
  gRNA of DRA-17
gacugucucugacacuccug

SEQ ID NO: 186 - RNA, 20 nt, Artificial Sequence
  gRNA of DRA-18
gagccucuucucaagcacug

SEQ ID NO: 187 - RNA, 20 nt, Artificial Sequence
  gRNA of DRA-19
gauaguggaacuugcggaaa

SEQ ID NO: 188 - RNA, 20 nt, Artificial Sequence
  gRNA of DRA-20
gaugagcgcucaggaaucau

SEQ ID NO: 189 - RNA, 20 nt, Artificial Sequence
  gRNA of DRA-21
gcuaucaaagguaggugcug

SEQ ID NO: 190 - RNA, 20 nt, Artificial Sequence
  gRNA of DRA-22
guuaccucuggagguacugg

SEQ ID NO: 191 - RNA, 20 nt, Artificial Sequence
  gRNA of DRA-23
uagcacagggacuccacuua

SEQ ID NO: 192 - RNA, 20 nt, Artificial Sequence
  gRNA of DRA-24
ugaugaaaaauccuagcaca

SEQ ID NO: 193 - RNA, 20 nt, Artificial Sequence
  gRNA of DRA-25
ugaugagcgcucaggaauca

SEQ ID NO: 194 - RNA, 20 nt, Artificial Sequence
  gRNA of DRA-27
uuugccagcuuugaggcuca

SEQ ID NO: 195 - RNA, 20 nt, Artificial Sequence
  gRNA of DRA-28
aacuauacuccgaucaccaa

SEQ ID NO: 196 - RNA, 20 nt, Artificial Sequence
  gRNA of DRA-29
agaagaacaugugaucaucc

SEQ ID NO: 197 - RNA, 20 nt, Artificial Sequence
  gRNA of DRA-30
agcagagagggagguaccau

SEQ ID NO: 198 - RNA, 20 nt, Artificial Sequence
  gRNA of DRA-31
agcgcuuugucaugauuucc

SEQ ID NO: 199 - RNA, 20 nt, Artificial Sequence
  gRNA of DRA-32
agcuguggacaaagccaacc

SEQ ID NO: 200 - RNA, 20 nt, Artificial Sequence
  gRNA of DRA-33
agggagguaccauuggugau

SEQ ID NO: 201 - RNA, 20 nt, Artificial Sequence
  gRNA of DRA-34
auaaacucgccugauugguc

SEQ ID NO: 202 - RNA, 20 nt, Artificial Sequence
  gRNA of DRA-35
auuggugaucggaguauagu

SEQ ID NO: 203 - RNA, 20 nt, Artificial Sequence
  gRNA of DRA-36
ccauguggauauggcaaaga

SEQ ID NO: 204 - RNA, 20 nt, Artificial Sequence
  gRNA of DRA-37
cuuugaggcucaaggugcau

SEQ ID NO: 205 - RNA, 20 nt, Artificial Sequence
  gRNA of DRA-38
cuuugucaugauuuccaggu

SEQ ID NO: 206 - RNA, 20 nt, Artificial Sequence
  gRNA of DRA-39
ggauauggcaaagaaggaga

SEQ ID NO: 207 - RNA, 20 nt, Artificial Sequence
  gRNA of DRA-40
uaucugaauccugaccaauc

SEQ ID NO: 208 - RNA, 20 nt, Artificial Sequence
  gRNA of DRA-41
ugagauuuuccauguggaua

SEQ ID NO: 209 - RNA, 20 nt, Artificial Sequence
  gRNA of DRA-42
ugaucacauguucuucugaa

SEQ ID NO: 210 - RNA, 20 nt, Artificial Sequence
  gRNA of DRA-43
ugcaccuugagccucaaagc

SEQ ID NO: 211 - RNA, 20 nt, Artificial Sequence
  gRNA of DRA-44
ugcauuggccaacauagcug

SEQ ID NO: 212 - RNA, 20 nt, Artificial Sequence
  gRNA of DRA-45
uggacgauuugccagcuuug

SEQ ID NO: 213 - RNA, 20 nt, Artificial Sequence
  gRNA of DRA-46
uggcaaagaaggagacgguc

SEQ ID NO: 214 - RNA, 20 nt, Artificial Sequence
  gRNA of DRA-47
uggugaugagauuuuccaug

SEQ ID NO: 215 - RNA, 20 nt, Artificial Sequence
  gRNA of DRA-48
aaugucacguggcuucgaaa

SEQ ID NO: 216 - RNA, 20 nt, Artificial Sequence
  gRNA of DRA-49
agacaaguucaccccaccag

SEQ ID NO: 217 - RNA, 20 nt, Artificial Sequence
  gRNA of DRA-50
agacaaguucaccccaccag

SEQ ID NO: 218 - RNA, 20 nt, Artificial Sequence
  gRNA of DRA-51
gaacgcagggggccucugua

SEQ ID NO: 219 - RNA, 20 nt, Artificial Sequence
  gRNA of DRA-52
cugaggacguuuacgacugc

SEQ ID NO: 220 - RNA, 20 nt, Artificial Sequence
  gRNA of DRA-53
gcggaaaagguggucuuccc

SEQ ID NO: 221 - RNA, 20 nt, Artificial Sequence
  gRNA of DRA-54
ggacguuuacgacugcaggg

SEQ ID NO: 222 - RNA, 20 nt, Artificial Sequence
  gRNA of DRA-55
gucguaaacguccucaguug

SEQ ID NO: 223 - RNA, 20 nt, Artificial Sequence
  gRNA of DRA-56
gugagcacaguuaccucugg

SEQ ID NO: 224 - RNA, 20 nt, Artificial Sequence
  gRNA of DRA-57
gugucccccaguaccuccag

SEQ ID NO: 225 - RNA, 20 nt, Artificial Sequence
  gRNA of DRA-58
ugaggacguuuacgacugca

SEQ ID NO: 226 - RNA, 20 nt, Artificial Sequence
  gRNA of DRA-59
aauggaaaaccugucaccac

SEQ ID NO: 227 - RNA, 20 nt, Artificial Sequence
  gRNA of DRA-60
aguggaacuugcggaaaagg

SEQ ID NO: 228 - RNA, 20 nt, Artificial Sequence
  gRNA of DRA-61
augaaacagaugaggacguu

SEQ ID NO: 229 - RNA, 20 nt, Artificial Sequence
  gRNA of DRA-62
cagagacagucuuccugccc

SEQ ID NO: 230 - RNA, 20 nt, Artificial Sequence
  gRNA of DRA-63
cgugacauugaccacuggug

SEQ ID NO: 231 - RNA, 20 nt, Artificial Sequence
  gRNA of DRA-64
uaugaaacagaugaggacgu

SEQ ID NO: 232 - RNA, 20 nt, Artificial Sequence
  gRNA of DRA-65
ucugacacuccuguggugac

SEQ ID NO: 233 - RNA, 20 nt, Artificial Sequence
  gRNA of DRA-66
aaacguccucaguugagggc

SEQ ID NO: 234 - RNA, 20 nt, Artificial Sequence
  gRNA of DRA-67
ucguaaacguccucaguuga

SEQ ID NO: 235 - protein, 20 aa, Artificial Sequence
  B2M signal peptide
MSRSVALAVLALLSLSGLEA

SEQ ID NO: 236 - protein, 9 aa, Artificial Sequence
  G-peptide
VMAPRTLFL

SEQ ID NO: 237 - protein, 99 aa, Artificial Sequence [sequence omitted]

SEQ ID NO: 238 - protein, 15 aa, Artificial Sequence
  G4S linker 1
GGGGSGGGGSGGGGS

SEQ ID NO: 239 - protein, 9 aa, Artificial Sequence
  HA tag
YPYDVPDYA

SEQ ID NO: 240 - protein, 337 aa, Artificial Sequence
  HLA-E
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 241 - protein, 20 aa, Artificial Sequence
  G4S linker 2
GGGGSGGGGSGGGGSGGGGS

SEQ ID NO: 242 - DNA, 20 bp, Artificial Sequence
  forward primer
ctggcttggagacaggtgac

SEQ ID NO: 243 - DNA, 20 bp, Artificial Sequence
  reverse primer
gacgcttatcgacgccctaa

SEQ ID NO: 244 - DNA, 21 bp, Artificial Sequence
  forward primer
cccaagtgaaataccctggca

SEQ ID NO: 245 - DNA, 20 bp, Artificial Sequence
  reverse primer
agcccttcctactagcctca

SEQ ID NO: 246 - DNA, 20 bp, Artificial Sequence
  forward primer
aatttcttggggagggggtg

SEQ ID NO: 247 - DNA, 24 bp, Artificial Sequence
  reverse primer
agctggatagtaggagaagacagt

SEQ ID NO: 248 - DNA, 22 bp, Artificial Sequence
  forward primer
gggttaaagagtctgtccgtga

SEQ ID NO: 249 - DNA, 23 bp, Artificial Sequence
  reverse primer
tgtcgagaccacataatacctgt

SEQ ID NO: 250 - DNA, 20 bp, Artificial Sequence
  forward primer
acctgacttggcagggtttg

SEQ ID NO: 251 - DNA, 21 bp, Artificial Sequence
  reverse primer
cccaagatctaccaccggaga

SEQ ID NO: 252 - DNA, 20 bp, Artificial Sequence
  forward primer
tgctcccaagcagaaggtaa

SEQ ID NO: 253 - DNA, 24 bp, Artificial Sequence
  reverse primer
aacccatgaagtgtggaaaacaag

SEQ ID NO: 254 - DNA, 20 bp, Artificial Sequence
  forward primer
tccctccataccagggttca

SEQ ID NO: 255 - DNA, 21 bp, Artificial Sequence
  reverse primer
aactcatccttaccccagtgt

SEQ ID NO: 256 - DNA, 20 bp, Artificial Sequence
  forward primer
tgtgtcaacttatgccgcgt

SEQ ID NO: 257 - DNA, 20 bp, Artificial Sequence
  reverse primer
ttgggaaacacggtcacctc

SEQ ID NO: 258 - DNA, 21 bp, Artificial Sequence
  forward primer
tgtgaactggagctctcttga

SEQ ID NO: 259 - DNA, 21 bp, Artificial Sequence
  reverse primer
tatgagggccagagggaacat

(19) United States
(12) Patent Application Publication
(10) Pub. No.: US 2020/0407713 A1
(43) Pub. Date: Dec. 31, 2020

(54) TRANSFORMED HUMAN CELL AND USE THEREOF

(71) Applicant: MOGAM INSTITUTE FOR BIOMEDICAL RESEARCH, Yongin-si, Gyeonggi-do (KR)

(72) Inventors: Ok Jae LIM, Yongin-si (KR); Mun Kyung KIM, Yongin-si (KR); Yun Jung LEE, Yongin-si (KR); Jee Won LEE, Yongin-si (KR); Woo Seok YANG, Yongin-si (KR); Yu Young KIM, Yongin-si (KR); Yongin Eun KWON, Yongin-si (KR); Seung Hyon CHOE, Yongin-si (KR)

(73) Assignee: MOGAM INSTITUTE FOR BIOMEDICAL RESEARCH, Yongin-si, Gyeonggi-do (KR)

(21) Appl. No.: 16/764,664

(22) PCT Filed: Nov. 16, 2018
(86) PCT No.: PCT/KR2018/014112

§ 371 Date: May 15, 2020

Related U.S. Application Data
(60) Provisional application No. 62/587,068, filed on Nov. 16, 2017.

(51) Int. Cl. C12N 15/11; C12N 9/22; A61K 35/17

(52) U.S. Cl. CPC C12N 15/11; C12N 2310/20; A61K 35/17; C12N 9/22

(57) ABSTRACT

The present invention relates to a transformed human cell and a use thereof and, more particularly, to a cell transformed with a gene for coding an MHC I cell membrane receptor and an MHC II cell membrane receptor by using a gene expression suppressing system using a guide RNA, and a use thereof. Such a transformed cell can effectively exhibit the therapeutic effect of cells even in vivo, and cannot be removed by an in vivo immune response. Therefore, it is expected that a composition comprising the immunocyte as an active ingredient can be usefully used for the treatment of cancer, infectious diseases, degenerative diseases or immunological diseases.

### TECHNICAL FIELD

The present invention relates to a transformed human cell and a use thereof, and more particularly, to a human cell transformed through a guide RNA and a use thereof.

### BACKGROUND ART

As a method for treating cancer or an infectious disease, immunotherapies using the patient's immune function are attracting attention. Immunotherapies mean treatment methods for diseases through interaction of immune cells such as NK cells, T cells, dendritic cells, and the like. Among these, immunotherapies are emerging which use genetically modified T cells expressing a chimeric antigen receptor specific for an antigen. In addition, it has been reported that NK cells, which are allowed to have high cytotoxicity by being activated ex vivo, exhibit an excellent therapeutic effect on blood cancer such as leukemia (Blood Cells Molecules & Disease, 33: p 261-266, 2004).

Meanwhile, despite possibility of immune cells as a therapeutic agent for cancer or an infectious disease as mentioned above, immune cells present in a patient's body are remarkably lower, in terms of function and number, as compared with those in healthy individuals. Therefore, it is more effective to utilize transplantation of allogeneic immune cells than to use autologous immune cells. However, in a case where allogeneic immune cells are transplanted, several problems may occur, such as transplant rejection, or immunological elimination caused by recognition of non-self in vivo. Accordingly, in order to overcome these drawbacks, there is a need for an alternative to making allogeneic immune cells into a cell banking while allowing the allogeneic immune cells to be recognized as self.

### DISCLOSURE OF INVENTION

### Technical Problem

In order to solve the above-mentioned problems, the present inventors have synthesized guide RNAs that target a gene encoding MHC I cell membrane receptor and a gene encoding MHC II cell membrane receptor in a cell. In addition, the present inventors have prepared a cell, in which expression of MHC I cell membrane receptor and MHC II cell membrane receptor is inhibited, using a composition for inhibiting gene expression which comprises, as active ingredients, the guide RNA and an RNA-guided endonuclease, wherein HLA-E may be introduced thereto so that in vivo immunological elimination to the cell is prevented.

Accordingly, an object of the present invention is to provide guide RNAs that target a gene encoding MHC I cell membrane receptor and a gene encoding MHC II cell membrane receptor, and to provide a cell transformed using the guide RNA.

### Solution to Problem

In order to achieve the above object, the present invention provides a guide RNA that complementarily binds to a nucleic acid sequence encoding P2-microglobulin (B2M), the guide RNA comprising any one nucleic acid sequence selected from the group consisting of SEQ ID NO: 1, SEQ ID NO: 6, SEQ ID NO: 17, and SEQ ID NO: 26; a guide RNA that complementarily binds to a nucleic acid sequence encoding HLA-DQ, the guide RNA comprising any one nucleic acid sequence selected from the group consisting of SEQ ID NO: 64, SEQ ID NO: 65, SEQ ID NO: 87, and SEQ ID NO: 90; a guide RNA that complementarily binds to a nucleic acid sequence encoding HLA-DP, the guide RNA comprising the nucleic acid sequence of SEQ ID NO: 123 or SEQ ID NO: 129; and a guide RNA that complementarily binds to a nucleic acid sequence encoding HLA-DR, the guide RNA comprising any one nucleic acid sequence selected from the group consisting of SEQ ID NO: 186, SEQ ID NO: 188, and SEQ ID NO: 225.

In addition, the present invention provides a composition for inhibiting gene expression comprising as active ingredients a guide RNA or a nucleotide sequence encoding the guide RNA, and an RNA-guided endonuclease or a nucleotide sequence encoding the RNA-guided endonuclease.

In addition, the present invention provides a transformed cell in which expression of MHC I cell membrane receptor and MHC II cell membrane receptor is inhibited.

In addition, the present invention provides a pharmaceutical composition for treating cancer, an infectious disease, a degenerative disease, a hereditary disease, or an immune disease, comprising the transformed cell as an active ingredient; and a method for treating cancer, an infectious disease, a degenerative disease, a hereditary disease, or an immune disease, comprising administering the composition to a subject.

In addition, the present invention provides a use of a transformed cell for treating cancer, an infectious disease, a degenerative disease, a hereditary disease, or an immune disease, wherein expression of MHC I cell membrane receptor and MHC II cell membrane receptor is inhibited in the transformed cell, and the transformed cell expresses a peptide antigen, such as G-peptide, bound to a modified MHC I cell membrane receptor on the cell membrane surface.

### Advantageous Effects of Invention

It is possible to prepare a cell in which a gene encoding MHC I cell membrane receptor and a gene encoding MHC II cell membrane receptor are modified, by using a gene expression inhibition system using a guide RNA according to the present invention. In addition, it is possible to additionally introduce, into the cell, HLA-E to which a peptide antigen such as G-peptide is bound. A cell transformed as described above can effectively show its therapeutic efficacy even in vivo, and is not eliminated by an in vivo immune response.

Therefore, it is expected that a composition comprising the cell as an active ingredient can be usefully used for the treatment of cancer, an infectious disease, a degenerative disease, a hereditary disease, or an immune disease.

### BRIEF DESCRIPTION OF DRAWINGS

FIG. 1 illustrates results obtained by analyzing, with flow cytometry, HLA-ABC negative cells in cells prepared by using a B2M-targeted gRNA.

FIG. 2 illustrates results obtained by analyzing, with flow cytometry, HLA-DR negative cells in cells prepared by using an HLA-DRA-targeted gRNA.

FIG. 3 illustrates results obtained by analyzing, with flow cytometry, HLA-DQ negative cells in cells prepared by using an HLA-DQA-targeted gRNA.

FIG. 4 illustrates results obtained by analyzing, with flow cytometry, HLA-DP negative cells in cells prepared by using an HLA-DPA-targeted gRNA.

FIG. 5 illustrates production rates of HLA-ABC negative cell line depending on B2M-targeted gRNAs.

FIG. 6 illustrates production rates of HLA-DR negative cell line depending on DRA-targeted gRNAs.

FIG. 7 illustrates production rates of HLA-DQ negative cell line depending on DQA-targeted gRNAs.

FIG. 8 illustrates production rates of HLA-DP negative cell line depending on DPA-targeted gRNAs.

FIG. 9 illustrates mutation in a nucleic acid encoding B2M in cell lines prepared with B2M-targeted gRNAs.

FIG. 10 illustrates mutation in a nucleic acid encoding HLA-DRA in cell lines prepared with HLA-DRA-targeted gRNAs.

FIG. 11 illustrates mutation in a nucleic acid encoding HLA-DQA in cell lines prepared with HLA-DQA-targeted gRNAs.

FIG. 12 illustrates mutation in a nucleic acid encoding HLA-DPA in cell lines prepared with HLA-DPA-targeted gRNAs.

FIG. 13 illustrates HLA-I positive NK-92MI cell line and HLA-I negative NK-92MI cell line after cell separation.

FIG. 14 illustrates evaluation results for cell-killing capacity of the HLA-I positive NK-92MI cell line and the HLA-I negative NK-92MI cell line.

FIG. 15 illustrates results obtained by transforming CD4 T cells, CD8 T cells, and NK cells using gRNAs and then performing analysis with flow cytometry.

FIG. 16 illustrates deletion efficiency for targets in single gRNA-transformed cells and multiple gRNA-transformed cells.

FIG. 17 compares cell growth rate among single gRNA-transformed cells, multiple gRNA-transformed cells, and control group cells.

FIG. 18 compares cytokine production capacity between HLA-I positive T cells and HLA-I negative T cells.

FIG. 19 compares cytokine production capacity between HLA-I positive NK cells and HLA-I negative NK cells.

FIG. 20 illustrates evaluation results for cell-killing capacity of NK cells against HLA-I positive Raji cell line and HLA-I negative Raji cell line.

FIG. 21 illustrates a schematic diagram of HLA-E loaded with G-peptide and a structure of a protein for expressing the same.

FIG. 22 illustrates results obtained by analyzing HLA-E expressed in K562 cell line through transduction.

FIG. 23 illustrates evaluation results for cell-killing capacity of NK cells against K562 cell line (K562 G-B2M-HLA-E) expressing HLA-E and control group K562 cell line (K562).

### BEST MODE FOR CARRYING OUT THE INVENTION

In an aspect of the present invention, there is provided a guide RNA that complementarily binds to a nucleic acid sequence encoding 2-microglobulin (B2M), the guide RNA comprising any one nucleic acid sequence selected from the group consisting of SEQ ID NO: 1, SEQ ID NO: 6, SEQ ID NO: 17, and SEQ ID NO: 26.

As used herein, the term “B2M” refers to P2-microglobulin protein that is a component of MHC I. B2M is essential for expression of MHC I cell membrane receptor on the cell surface; and when B2M is removed or modified, expression of the MHC I cell membrane receptor on the cell surface is difficult to occur. Thus, the function of the MHC I cell membrane receptor may be removed by modifying the gene of B2M.

The guide RNA that complementarily binds to a nucleic acid sequence encoding B2M may be any one selected from the group consisting of SEQ ID NOs: 1 to 58, and may specifically be any one selected from the group consisting of SEQ ID NO: 1, SEQ ID NO: 6, SEQ ID NO: 17, and SEQ ID NO: 26.

In addition, in an aspect of the present invention, there is provided a guide RNA that complementarily binds to a nucleic acid sequence encoding HLA-DQ, the guide RNA comprising any one nucleic acid sequence selected from the group consisting of SEQ ID NO: 64, SEQ ID NO: 65, SEQ ID NO: 87, and SEQ ID NO: 90.

As used herein, the term “HLA” refers to a human leukocyte antigen that is a product of MHC gene. HLA is composed of HLA I and HLA II. HLA I may include HLA-A, HLA-B, and HLA-C; and HLA II may include HLA-DQ, HLA-DP, and HLA-DR.

As used herein, the term “HLA-DQ” refers to an αβ heterodimer constituting MHC II. DQ consists of HLA-DQA1 and HLA-DQB1. The a subunit is encoded by HLA-DQA1 gene, and the R subunit is encoded by HLA-DQB1 gene. Expression of MHC II cell membrane receptor may be inhibited by modifying the gene of DQ.

The guide RNA that complementarily binds to a nucleic acid sequence encoding DQ may be any one selected from the group consisting of SEQ ID NOs: 59 to 116, and may specifically be any one selected from the group consisting of SEQ ID NO: 64, SEQ ID NO: 65, SEQ ID NO: 87, and SEQ ID NO: 90.

In another aspect of the present invention, there is provided a guide RNA that complementarily binds to a nucleic acid sequence encoding HLA-DP, the guide RNA comprising the nucleic acid sequence of SEQ ID NO: 123 or SEQ ID NO: 129.

As used herein, the term “HLA-DP” refers to an encoded MHC II cell surface receptor that consists of DPα subunit and DPβ subunit. DPa is encoded by HLA-DPA1, and DPβ is encoded by HLA-DPBL. Expression of MHC II cell membrane receptor may be inhibited by modifying the gene of DP.

The guide RNA that complementarily binds to a nucleic acid sequence encoding DP may be any one selected from the group consisting of SEQ ID NOs: 117 to 175, and may specifically be SEQ ID NO: 123 or SEQ ID NO: 129.

In addition, in yet another aspect of the present invention, there is provided a guide RNA that complementarily binds to a nucleic acid sequence encoding HLA-DR, the guide RNA comprising any one nucleic acid sequence selected from the group consisting of SEQ ID NO: 186, SEQ ID NO: 188, and SEQ ID NO: 225.

As used herein, the term “HLA-DR” refers to an MHC II cell surface receptor, specifically an αβ heterodimer that constitutes the MHC II cell surface receptor. Each subunit of HLA-DR contains two extracellular domains, a membrane-spanning domain and a cytoplasmic tail. Expression of MHC II cell membrane receptor may be inhibited by modifying the gene of DR.

The guide RNA that complementarily binds to a nucleic acid sequence encoding DR may be any one selected from the group consisting of SEQ ID NOs: 176 to 234, and may preferably be any one selected from the group consisting of SEQ ID NO: 186, SEQ ID NO: 188, and SEQ ID NO: 225.

As used herein, the term “guide RNA (gRNA)” refers to an RNA molecule that specifically recognizes a target DNA and forms a complex with a nuclease, thereby guiding the nuclease to the target DNA.

The guide RNA may be a guide RNA derived from a prokaryotic clustered regularly interspaced short palindromic repeats (CRISPR) system.

The guide RNA may contain a non-naturally occurring chimeric crRNA sequence, and the crRNA sequence may contain a variable targeting domain capable of hybridizing to a target sequence.

In addition, the guide RNA contains a complementary sequence for each of B2M, HLA-DQ, HLA-DP, and HLA-DR genes. After being delivered into a cell, the guide RNA is capable of recognizing the target sequence and forming a complex with an RNA-guided endonuclease.

In yet another aspect of the present invention, there is provided a composition for inhibiting gene expression, comprising as active ingredients, the guide RNA or a nucleotide sequence encoding the guide RNA, and an RNA-guided endonuclease or a nucleotide sequence encoding the RNA-guided endonuclease.

The RNA-guided endonuclease may be delivered in the form of mRNA or protein, or may be delivered to a target cell by transformation using a vector loaded with DNA encoding the same. When an endonuclease in the form of protein is used, the endonuclease may function as an RNP complex obtained by forming a complex with the guide RNA.

As used herein, the term “RNP complex” refers to a complex that comprises, as active ingredients, the guide RNA and the RNA-guided endonuclease, wherein the complex is capable of recognizing and binding to a target sequence, thereby selectively nicking or cleaving the target sequence. The RNA complex may be, for example, a Cas9-gRNA complex but is not limited thereto.

In an embodiment of the present invention, the RNA-guided endonuclease may be any one selected from the group consisting of Cas1, Cas1B, Cas2, Cas3, Cas4, Cas5, Cas6, Cas7, Cas8, Cas9, Cas10, Cas12a, Cas12b, Cas12c, Cas12d, Cas12e, Cas 13a, Cas 13b, Cas 13c, Cas 13d, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, and may specifically be Cas9.

In an aspect of the present invention, there is provided a transformed cell in which expression of MHC I cell membrane receptor and MHC II cell membrane receptor is inhibited.

As used herein, the term “expression inhibition” means modification on a nucleotide sequence which causes a decrease in the function of a target gene, and preferably means that expression of a target gene is made undetectable or the target gene is expressed to a meaningless level, due to such expression inhibition.

In an embodiment of the present invention, the transformed cell may express a peptide antigen on the cell membrane surface. Examples of the peptide antigen include, but are not limited to, signal peptides of HLA-A, HLA-B, HLA-C, and HLA-G, and the peptide antigen is specifically a signal peptide (G-peptide) of HLA-G. The peptide antigen may be bound to modified MHC I cell membrane receptor.

In an embodiment of the present invention, the modified MHC I cell membrane receptor has a structure in which HLA-E and B2M are linked.

Specifically, the C-terminus of B2M may be linked, via a first linker, to the N-terminus of al of HLA-E and the C-terminus of G-peptide may be linked, via a second linker, to the N-terminus of B2M in the modified MHC I cell membrane receptor. The modified MHC I cell membrane receptor may have a structure in which HLA-G and B2M are linked.

In an embodiment of the present invention, G-peptide may have the sequence of SEQ ID NO: 236; HLA-E may have the sequence of SEQ ID NO: 240; B2M may have the sequence of SEQ ID NO: 237; and the first linker may be (G₄S)ₙ (n is an integer of 1 to 5) and may have the sequence of SEQ ID NO: 238 in an embodiment. The second linker may be (G₄S)ₙ (n is an integer of 2 to 6) and may have the sequence of SEQ ID NO: 241.

In an embodiment of the present invention, modification of a gene encoding the MHC I cell membrane receptor may be performed using the guide RNA (for example, SEQ ID NO: 1, SEQ ID NO: 6, SEQ ID NO: 17, or SEQ ID NO: 26) that complementarily binds to a nucleic acid sequence encoding B2M. Specifically, the modification of MHC I may be performed by single deletion using a single guide RNA.

In an embodiment of the present invention, modification of DQ, DP, and DR genes encoding the MHC II cell membrane receptor may be performed using the guide RNA (for example, SEQ ID NO: 64, SEQ ID NO: 65, SEQ ID NO: 87, or SEQ ID NO: 90) that complementarily binds to a nucleic acid sequence encoding DQ, the guide RNA (for example, SEQ ID NO: 123 or SEQ ID NO: 129) that complementarily binds to a nucleic acid sequence encoding DP, and the guide RNA (for example, SEQ ID NO: 186, SEQ ID NO: 188, or SEQ ID NO: 225) that complementarily binds to a nucleic acid sequence encoding DR. The modification of MHC II is performed together with the modification of MHC I, which may be performed by multiplex deletion using a multiple guide RNA (such as containing all of SEQ ID NO: 1, SEQ ID NO: 64, SEQ ID NO: 129, and SEQ ID NO: 188).

In an embodiment of the present invention, the transformed cell may be a therapeutic allogeneic cell. As used herein, the term “therapeutic allogeneic cell” refers to a non-autologous allogeneic cell to be injected into a subject for the purpose of suppressing progression of, treating, or alleviating symptoms of a disease, and examples thereof include, but are not limited to, immune cells and stem cells.

As used herein, the term “immune cell” refers to a cell involved in immune responses of the human body, and examples thereof include NK cells, T cells, B cells, dendritic cells, and macrophages.

In an embodiment of the present invention, the immune cell may be an NK cell or T cell.

As used herein, the term “stem cell” refers to a pluripotent cell capable of being differentiated into various cells. Examples of the stem cell may include human embryonic stem cells, bone marrow stem cells, mesenchymal stem cells, human nerve stem cells, oral mucosal cells, and the like. Specifically, the stem cell may be a mesenchymal stem cell.

In addition, in an aspect of the present invention, there is provided a pharmaceutical composition for treating cancer, an infectious disease, a degenerative disease, a hereditary disease, or an immune disease, comprising the transformed cell as an active ingredient.

In an embodiment of the present invention, the cancer may be any one selected from the group consisting of chronic lymphocytic leukemia (CLL), B-cell acute lymphocytic leukemia (B-ALL), acute lymphoblastic leukemia, acute myeloid leukemia, lymphoma, non-Hodgkin's lymphoma (NHL), multiple myeloma, blood cancer, gastric cancer, liver cancer, pancreatic cancer, colorectal cancer, lung cancer, breast cancer, ovarian cancer, skin cancer, melanoma, sarcoma, prostate cancer, esophageal cancer, hepatocellular carcinoma, astrocytoma, mesothelioma, head and neck cancer, and medulloblastoma.

In an embodiment of the present invention, the infectious disease may be any one selected from the group consisting of hepatitis B, hepatitis C, human papilloma virus (HPV) infection, cytomegalovirus infection, Epstein Barr virus (EBV) infection, viral respiratory disease, and influenza.

As used herein, the term “degenerative disease” refers to a pathological condition in which a tissue loses its original function due to irreversible quantitative loss of the tissue. Examples of the degenerative disease include, but are not limited to, brain neurological disease, ischemic disease, skin damage, bone disease, and degenerative arthritis.

As used herein, the term “hereditary disease” refers to a pathological condition that occurs due to a mutation that is harmful to a gene or chromosome. Examples of the hereditary disease include, but are not limited to, hemophilia, albinism, Fabry disease, Hunter syndrome, and glycogen storage disorder.

As used herein, the term “immune disease” refers to any pathological condition in which a tissue is damaged due to an excessive or undesired immune response. Accordingly, the term “immune disease” has the same meaning as “hyperactive immune disease”, and the term “composition for preventing or treating an immune disease” has the same meaning as “immunosuppressant”.

Examples of the immune disease include, but are not limited to, graft-versus-host disease, graft rejection, chronic inflammatory disease, inflammatory pain, neuropathic pain, chronic obstructive pulmonary disease (COPD), and autoimmune disease.

The term “autoimmune disease” refers to a pathological condition that occurs when immune cells fail to distinguish self from a foreign substance and thus attack the self Examples of the autoimmune disease may include, but are not limited to, rheumatoid arthritis, systemic lupus erythematosis, Hashimoto's thyroiditis, Grave's disease, multiple sclerosis, scleroderma, myasthenia gravis, type I diabetes, allergic encephalomyelitis, glomerulonephritis, vitiligo, Behcet's disease, Crohn's disease, ankylosing spondylitis, thrombocytopenic purpura, pemphigus vulgaris, autoimmune hemolytic anemia, adrenoleukodystrophy (ALD), and systemic lupus erythematosus (SLE).

In an aspect of the present invention, there is provided a method for treating cancer, an infectious disease, a degenerative disease, a hereditary disease, or an immune disease, comprising administering the pharmaceutical composition to a subject.

In an embodiment of the present invention, the administration may be performed via any one route selected from the group consisting of intravenous, intramuscular, intradermal, subcutaneous, intraperitoneal, intraarteriolar, intraventricular, intralesional, intrathecal, topical, and combinations thereof.

In another aspect of the present invention, there is provided a use of a transformed cell for treating cancer, an infectious disease, a degenerative disease, a hereditary disease, or an immune disease, wherein expression of MHC I cell membrane receptor and MHC II cell membrane receptor is inhibited in the transformed cell, and the transformed cell expresses G-peptide bound to modified MHC I cell membrane receptor on the cell membrane surface.

In yet another aspect of the present invention, there is provided a kit for modifying a gene for MHC I cell membrane receptor and a gene for MHC II cell membrane receptor, the kit comprising the guide RNA or a nucleotide sequence encoding a guide RNA, and an RNA-guided endonuclease or a nucleotide sequence encoding the RNA-guided endonuclease.

### MODE FOR THE INVENTION

Hereinafter, the present invention will be described in more detail by way of the following examples. However, the following examples are only for illustrating the present invention, and the scope of the present invention is not limited thereto.

### Example 1. Synthesis and Selection of gRNA Targeting HLA

### Example 1.1. Search and Synthesis of gRNA Sequence

In order to search for a gRNA sequence, the complete nucleotide sequences of genes provided by NCBI (https://www.ncbi.nlm.nih.gov/) were used. As design tools for gRNAs, web-based systems, CHOPCHOP (http://chopchop.cbu.uib.no/), E-CRISP (http://www.e-crisp.org/E-CRISP/designcrispr.html), CRISPR-ERA (http://crispr-era.stanford.edu/), RGEN Tools (http://www.rgenome.net/cas-designer/) were used. Among the designed gRNAs, about 60 gRNAs, which were most suitable for gene knock-out, were obtained per each desired target. Based on these sequences, gRNAs were synthesized using the GeneArt Precision gRNA Synthesis Kit (Thermo Fisher Scientific, A29377) according to the manufacturer's instructions.

That is, forward and reverse oligonucleotide primers, required to synthesize a DNA template encoding each of the gRNAs, were synthesized, and then PCR was performed with a PCR thermal cycler (FlexCycler2, Analytik Jena) using the synthesized primers and Tracr Fragment+T7 Primer Mix contained in the GeneArt Precision gRNA Synthesis Kit (Thermo Fisher Scientific, A29377). The following PCR parameters were used: pre-denaturation at 98° C. for 10 seconds, followed by 32 cycles of denaturation and annealing under a condition of at 98° C. for 5 seconds and at 55° C. for 15 seconds, followed by final extension at 72° C. for 1 minute. Using the obtained PCR product as a template, an in vitro transcription reaction was performed at 37° C. for 4 hours; and then the resultant was purified to obtain a gRNA.

The obtained gRNAs are shown in Tables 1 to 4 below. Specifically, the gRNA sequences for HLA-ABC (B2M) are shown in Table 1; the gRNA sequences for HLA-DQ are shown in Table 2; the gRNA sequences for HLA-DP are shown in Table 3 below; and the gRNA sequences for HLA-DR are shown in Table 4 below.

TABLE 1

| HLA-ABC | gRNA sequence | SEQ ID NO |
|---|---|---|
| B2M-01 | GAGUAGCGCGAGCACAGCUA | SEQ ID NO: 1 |
| B2M-03 | CUCGCGCUACUCUCUCUUUC | SEQ ID NO: 2 |
| B2M-04 | GCAUACUCAUCUUUUUCAGU | SEQ ID NO: 3 |
| B2M-05 | GCUACUCUCUCUUUCUGGCC | SEQ ID NO: 4 |
| B2M-06 | GGCAUACUCAUCUUUUUCAG | SEQ ID NO: 5 |
| B2M-07 | GGCCACGGAGCGAGACAUCU | SEQ ID NO: 6 |
| B2M-08 | GGCCGAGAUGUCUCGCUCCG | SEQ ID NO: 7 |
| B2M-09 | UCACGUCAUCCAGCAGAGAA | SEQ ID NO: 8 |
| B2M-10 | ACAAAGUCACAUGGUUCACA | SEQ ID NO: 9 |
| B2M-11 | AGUCACAUGGUUCACACGGC | SEQ ID NO: 10 |
| B2M-12 | AAGUCAACUUCAAUGUCGGA | SEQ ID NO: 11 |
| B2M-13 | CAUACUCAUCUUUUUCAGUG | SEQ ID NO: 12 |
| B2M-14 | UCCUGAAUUGCUAUGUGUCU | SEQ ID NO: 13 |
| B2M-15 | CGUGAGUAAACCUGAAUCUU | SEQ ID NO: 14 |
| B2M-16 | UUGGAGUACCUGAGGAAUAU | SEQ ID NO: 15 |
| B2M-17 | AGGGUAGGAGAGACUCACGC | SEQ ID NO: 16 |
| B2M-18 | ACAGCCCAAGAUAGUUAAGU | SEQ ID NO: 17 |
| B2M-19 | AUACUCAUCUUUUUCAGUGG | SEQ ID NO: 18 |
| B2M-20 | UGGAGUACCUGAGGAAUAUC | SEQ ID NO: 19 |
| B2M-21 | AAGAAAAGGAAACUGAAAAC | SEQ ID NO: 20 |
| B2M-22 | AAGAAGGCAUGCACUAGACU | SEQ ID NO: 21 |
| B2M-23 | ACAUGUAAGCAGCAUCAUGG | SEQ ID NO: 22 |
| B2M-24 | ACCCAGACACAUAGCAAUUC | SEQ ID NO: 23 |
| B2M-25 | ACUUGUCUUUCAGCAAGGAC | SEQ ID NO: 24 |
| B2M-26 | CAAGCCAGCGACGCAGUGCC | SEQ ID NO: 25 |
| B2M-27 | CACAGCCCAAGAUAGUUAAG | SEQ ID NO: 26 |
| B2M-29 | CAUCACGAGACUCUAAGAAA | SEQ ID NO: 27 |
| B2M-30 | CGCAGUGCCAGGUUAGAGAG | SEQ ID NO: 28 |
| B2M-31 | CUAACCUGGCACUGCGUCGC | SEQ ID NO: 29 |
| B2M-32 | GAAAGUCCCUCUCUCUAACC | SEQ ID NO: 30 |
| B2M-33 | GAGACAUGUAAGCAGCAUCA | SEQ ID NO: 31 |
| B2M-34 | GAGUCUCGUGAUGUUUAAGA | SEQ ID NO: 32 |
| B2M-35 | GCAGUGCCAGGUUAGAGAGA | SEQ ID NO: 33 |
| B2M-36 | UAAGAAGGCAUGCACUAGAC | SEQ ID NO: 34 |
| B2M-37 | UCGAUCUAUGAAAAAGACAG | SEQ ID NO: 35 |
| B2M-39 | UUCAGACUUGUCUUUCAGCA | SEQ ID NO: 36 |
| B2M-40 | UUCCUGAAUUGCUAUGUGUC | SEQ ID NO: 37 |
| B2M-41 | UAAGAAAAGGAAACUGAAAA | SEQ ID NO: 38 |
| B2M-42 | CUGGCACUGCGUCGCUGGCU | SEQ ID NO: 39 |
| B2M-43 | UGCGUCGCUGGCUUGGAGAC | SEQ ID NO: 40 |
| B2M-44 | GCUGGCUUGGAGACAGGUGA | SEQ ID NO: 41 |
| B2M-45 | AGACAGGUGACGGUCCCUGC | SEQ ID NO: 42 |
| B2M-46 | CAAUCAGGACAAGGCCCGCA | SEQ ID NO: 43 |
| B2M-47 | CCUGCGGGCCUUGUCCUGAU | SEQ ID NO: 44 |
| B2M-48 | CCAAUCAGGACAAGGCCCGC | SEQ ID NO: 45 |
| B2M-49 | CGGGCCUUGUCCUGAUUGGC | SEQ ID NO: 46 |
| B2M-50 | GGGCCUUGUCCUGAUUGGCU | SEQ ID NO: 47 |
| B2M-51 | GUGCCCAGCCAAUCAGGACA | SEQ ID NO: 48 |
| B2M-52 | AAACGCGUGCCCAGCCAAUC | SEQ ID NO: 49 |
| B2M-53 | GGGCACGCGUUUAAUAUAAG | SEQ ID NO: 50 |
| B2M-54 | CACGCGUUUAAUAUAAGUGG | SEQ ID NO: 51 |
| B2M-55 | UAUAAGUGGAGGCGUCGCGC | SEQ ID NO: 52 |
| B2M-56 | AAGUGGAGGCGUCGCGCUGG | SEQ ID NO: 53 |
| B2M-57 | AGUGGAGGCGUCGCGCUGGC | SEQ ID NO: 54 |
| B2M-58 | UUCCUGAAGCUGACAGCAUU | SEQ ID NO: 55 |
| B2M-59 | UCCUGAAGCUGACAGCAUUC | SEQ ID NO: 56 |
| B2M-60 | UGGGCUGUGACAAAGUCACA | SEQ ID NO: 57 |
| B2M-61 | ACUCUCUCUUUCUGGCCUGG | SEQ ID NO: 58 |

TABLE 2

| HLA-DQ | gRNA sequence | SEQ ID NO |
|---|---|---|
| DQA-08 | UUAGGAUCAUCCUCUUCCCA | SEQ ID NO: 59 |
| DQA-09 | AACUCUACCGCUGCUACCAA | SEQ ID NO: 60 |
| DQA-10 | ACAAUGUCUUCACCUCCACA | SEQ ID NO: 61 |
| DQA-11 | ACCACCGUGAUGAGCCCCUG | SEQ ID NO: 62 |
| DQA-12 | ACCCAGUGUCACGGGAGACU | SEQ ID NO: 63 |
| DQA-14 | ACCUCCACAGGGGCUCAUCA | SEQ ID NO: 64 |
| DQA-15 | CAAUGUCUUCACCUCCACAG | SEQ ID NO: 65 |
| DQA-16 | CACAAUGUCUUCACCUCCAC | SEQ ID NO: 66 |
| DQA-17 | CAGUACACCCAUGAAUUUGA | SEQ ID NO: 67 |
| DQA-18 | CUCUGUGAGCUCUGACAUAG | SEQ ID NO: 68 |
| DQA-19 | CUGUGGAGGUGAAGACAUUG | SEQ ID NO: 69 |
| DQA-20 | GGCUGGAAUCUCAGGCUCUG | SEQ ID NO: 70 |
| DQA-21 | GUUGGGCUGACCCAGUGUCA | SEQ ID NO: 71 |
| DQA-22 | UCAUGGGUGUACUGGCCAGA | SEQ ID NO: 72 |
| DQA-23 | UCCAAGUCUCCCGUGACACU | SEQ ID NO: 73 |
| DQA-24 | UCCACAGGGGCUCAUCACGG | SEQ ID NO: 74 |
| DQA-25 | UGUGGAGGUGAAGACAUUGU | SEQ ID NO: 75 |
| DQA-26 | UUCCAAGUCUCCCGUGACAC | SEQ ID NO: 76 |
| DQA-27 | UUGGGCUGACCCAGUGUCAC | SEQ ID NO: 77 |
| DQA-28 | AACAUCACAUGGCUGAGCAA | SEQ ID NO: 78 |
| DQA-29 | ACAUCACAUGGCUGAGCAAU | SEQ ID NO: 79 |
| DQA-30 | AGCCAUGUGAUGUUGACCAC | SEQ ID NO: 80 |
| DQA-31 | AGGAAUGAUCACUCUUGGAG | SEQ ID NO: 81 |
| DQA-32 | AUCACUCUUGGAGAGGAAGC | SEQ ID NO: 82 |
| DQA-33 | AUGACUGCAAGGUGGAGCAC | SEQ ID NO: 83 |
| DQA-34 | CAAGGUGGAGCACUGGGGCC | SEQ ID NO: 84 |
| DQA-35 | CAUCAAAUUCAUGGGUGUAC | SEQ ID NO: 85 |
| DQA-36 | CAUGUGAUGUUGACCACAGG | SEQ ID NO: 86 |
| DQA-37 | CCUCACCACAGAGGUUCCUG | SEQ ID NO: 87 |
| DQA-38 | CUCAUCUCCAUCAAAUUCAU | SEQ ID NO: 88 |
| DQA-39 | CUCCUGUGGUCAACAUCACA | SEQ ID NO: 89 |
| DQA-40 | GAAGAAGGAAUGAUCACUCU | SEQ ID NO: 90 |
| DQA-41 | GACUGCAAGGUGGAGCACUG | SEQ ID NO: 91 |
| DQA-42 | GAGGUAACUGAUCUUGAAGA | SEQ ID NO: 92 |
| DQA-43 | GGACAACAUCUUUCCUCCUG | SEQ ID NO: 93 |
| DQA-44 | GUGCUGUUUCCUCACCACAG | SEQ ID NO: 94 |
| DQA-45 | UCUUCUGAAACACUGGGGUA | SEQ ID NO: 95 |
| DQA-47 | UUCAUGGGUGUACUGGCCAG | SEQ ID NO: 96 |
| DQA-48 | AGAGACUGUGGUCUGCGCCC | SEQ ID NO: 97 |
| DQA-49 | GACAUAGGGGCUGGAAUCUC | SEQ ID NO: 98 |
| DQA-50 | GAGACUGUGGUCUGCGCCCU | SEQ ID NO: 99 |
| DQA-51 | GGCCUCGUGGGCAUUGUGGU | SEQ ID NO: 100 |
| DQA-52 | GGGCCUCGUGGGCAUUGUGG | SEQ ID NO: 101 |
| DQA-53 | GUCAGAGCUCACAGAGACUG | SEQ ID NO: 102 |
| DQA-54 | GUCUCUGUGAGCUCUGACAU | SEQ ID NO: 103 |
| DQA-55 | GUGAGCUCUGACAUAGGGGC | SEQ ID NO: 104 |
| DQA-56 | GUUGGUGCUUCCAGACACCA | SEQ ID NO: 105 |
| DQA-57 | UCUCUGUGAGCUCUGACAUA | SEQ ID NO: 106 |
| DQA-58 | UGACUGCAAGGUGGAGCACU | SEQ ID NO: 107 |
| DQA-59 | UGCCCACCACAAUGCCCACG | SEQ ID NO: 108 |
| DQA-60 | UGGAAGCACCAACUGAACGC | SEQ ID NO: 109 |
| DQA-61 | UGUGGGCCUCGUGGGCAUUG | SEQ ID NO: 110 |
| DQA-62 | UUACCCCAGUGUUUCAGAAG | SEQ ID NO: 111 |
| DQA-63 | UUGGAAAACACUGUGACCUC | SEQ ID NO: 112 |
| DQA-64 | UUGGUGCUUCCAGACACCAA | SEQ ID NO: 113 |
| DQA-65 | AAACAAAGCUCUGCUGCUGG | SEQ ID NO: 114 |
| DQA-66 | AAAUCUCAUCAGCAGAAGGG | SEQ ID NO: 115 |
| DQA-67 | CUAAACAAAGCUCUGCUGCU | SEQ ID NO: 116 |

TABLE 3

| HLA-DP | gRNA sequence | SEQ ID NO |
|---|---|---|
| DPA-01 | UCUAUGCGUCUGUACAAACG | SEQ ID NO: 117 |
| DPA-02 | GUACAGACGCAUAGACCAAC | SEQ ID NO: 118 |
| DPA-03 | UACAGACGCAUAGACCAACA | SEQ ID NO: 119 |
| DPA-04 | GAAGGAGACCGUCUGGCAUC | SEQ ID NO: 120 |
| DPA-05 | GGAGACCGUCUGGCAUCUGG | SEQ ID NO: 121 |
| DPA-06 | GUCUGGCAUCUGGAGGAGUU | SEQ ID NO: 122 |
| DPA-07 | GUGGUUGGAACGCUGGAUCA | SEQ ID NO: 123 |
| DPA-08 | GUCUUCAGGGCGCAUGUUGU | SEQ ID NO: 124 |
| DPA-09 | UGUCUUCAGGGCGCAUGUUG | SEQ ID NO: 125 |
| DPA-10 | UCUUCAGGGCGCAUGUUGUG | SEQ ID NO: 126 |
| DPA-11 | GUUGCAUACCCCAGUGCUUG | SEQ ID NO: 127 |
| DPA-12 | GACCUUUGUGCCCUCAGCAG | SEQ ID NO: 128 |
| DPA-13 | GAGACUCAGCAGGAAAGCCA | SEQ ID NO: 129 |
| DPA-14 | GAGCCUCAAAGGAAAAGGCU | SEQ ID NO: 130 |
| DPA-15 | GAUCUUGAGAGCCCUCUCCU | SEQ ID NO: 131 |
| DPA-16 | GCCAUCAAGGGUGAGUGCUC | SEQ ID NO: 132 |
| DPA-17 | GCCAUGACCCCCGGGCCCAG | SEQ ID NO: 133 |
| DPA-18 | GCCCAGCUCCACAGGCUCCU | SEQ ID NO: 134 |
| DPA-19 | GCCCUGAGCCUCAAAGGAAA | SEQ ID NO: 135 |
| DPA-20 | GCCUUUUCCUUUGAGGCUCA | SEQ ID NO: 136 |
| DPA-21 | GCGUUCUGGCCAUGACCCCC | SEQ ID NO: 137 |
| DPA-22 | GCUUUCCUGCUGAGUCUCCG | SEQ ID NO: 138 |
| DPA-23 | GGAAACACGGUCACCUCAGG | SEQ ID NO: 139 |
| DPA-24 | GGACUUCUAUGACUGCAGGG | SEQ ID NO: 140 |
| DPA-25 | GGAGACUGUGCUCUGUGCCC | SEQ ID NO: 141 |
| DPA-26 | GGCCAUGACCCCCGGGCCCA | SEQ ID NO: 142 |
| DPA-27 | GGCCUAGUCGGCAUCAUCGU | SEQ ID NO: 143 |
| DPA-29 | GGGAAACACGGUCACCUCAG | SEQ ID NO: 144 |
| DPA-30 | GGGCCUAGUCGGCAUCAUCG | SEQ ID NO: 145 |
| DPA-31 | GUCAUAGAAGUCCUCUGCUG | SEQ ID NO: 146 |
| DPA-32 | GUCCUCUGCUGAGGGCACAA | SEQ ID NO: 147 |
| DPA-33 | GUGGAAGCUGUAAUCUGUUC | SEQ ID NO: 148 |
| DPA-34 | GUGGGAAGAACUUGUCAAUG | SEQ ID NO: 149 |
| DPA-35 | GUUGGUGGCCUGAGUGUGGU | SEQ ID NO: 150 |
| DPA-36 | GUUGUCUCAGGCAUCUGGAU | SEQ ID NO: 151 |
| DPA-37 | GCUGAGUCUCCGAGGAGCUG | SEQ ID NO: 152 |
| DPA-38 | UCUCUACUGUCUUUAUGCAG | SEQ ID NO: 153 |
| DPA-39 | UAUGGAACAUUCUGUCUUCA | SEQ ID NO: 154 |
| DPA-40 | UCAAGAUCACAGCUCUGAUA | SEQ ID NO: 155 |
| DPA-41 | UCAAACAUAAACUCCCCUGU | SEQ ID NO: 156 |
| DPA-42 | UACCGUUGGUGGCCUGAGUG | SEQ ID NO: 157 |
| DPA-43 | UCCUGAGCACUCACCCUUGA | SEQ ID NO: 158 |
| DPA-44 | UGAGGUGACCGUGUUUCCCA | SEQ ID NO: 159 |
| DPA-45 | UGCGUUCUGGCCAUGACCCC | SEQ ID NO: 160 |
| DPA-46 | UUUCCUUUGAGGCUCAGGGC | SEQ ID NO: 161 |
| DPA-47 | UGCCGACUAGGCCCAGCACC | SEQ ID NO: 162 |
| DPA-48 | UCAGCAGGAAAGCCAAGGAG | SEQ ID NO: 163 |
| DPA-49 | UGAAGAUGAGAUGUUCUAUG | SEQ ID NO: 164 |
| DPA-50 | UGCUGAGUCUCCGAGGAGCU | SEQ ID NO: 165 |
| DPA-51 | UGAGAUGUUCUAUGUGGAUC | SEQ ID NO: 166 |
| DPA-52 | UGGGAAACACGGUCACCUCA | SEQ ID NO: 167 |
| DPA-53 | UGGAAGCUGUAAUCUGUUCU | SEQ ID NO: 168 |
| DPA-54 | UGGACAAGAAGGAGACCGUC | SEQ ID NO: 169 |
| DPA-55 | UGCCCACGAUGAUGCCGACU | SEQ ID NO: 170 |
| DPA-56 | UGGCCAAGCCUUUUCCUUUG | SEQ ID NO: 171 |
| DPA-57 | GUGGCUGUGCAACGGGGAGC | SEQ ID NO: 172 |
| DPA-58 | UCCCCUGGGCCCGGGGGUCA | SEQ ID NO: 173 |
| DPA-59 | UCACCUCAGGGGGAUCUGGA | SEQ ID NO: 174 |
| DPA-60 | UCUCCUUCCAGAUCCCCCUG | SEQ ID NO: 175 |

TABLE 4

| HLA-DR | gRNA sequence | SEQ ID NO |
|---|---|---|
| DRA-08 | AAGAAGAAAAUGGCCAUAAG | SEQ ID NO: 176 |
| DRA-09 | AAUCAUGGGCUAUCAAAGGU | SEQ ID NO: 177 |
| DRA-10 | AGCUGUGCUGAUGAGCGCUC | SEQ ID NO: 178 |
| DRA-11 | AUAAGUGGAGUCCCUGUGCU | SEQ ID NO: 179 |
| DRA-12 | ACUUAUGGCCAUUUUCUUCU | SEQ ID NO: 180 |
| DRA-13 | AUGAUGAAAAAUCCUAGCAC | SEQ ID NO: 181 |
| DRA-14 | CAGAGCGCCCAAGAAGAAAA | SEQ ID NO: 182 |
| DRA-15 | CAGGAAUCAUGGGCUAUCAA | SEQ ID NO: 183 |
| DRA-16 | CUUAUGGCCAUUUUCUUCUU | SEQ ID NO: 184 |
| DRA-17 | GACUGUCUCUGACACUCCUG | SEQ ID NO: 185 |
| DRA-18 | GAGCCUCUUCUCAAGCACUG | SEQ ID NO: 186 |
| DRA-19 | GAUAGUGGAACUUGCGGAAA | SEQ ID NO: 187 |
| DRA-20 | GAUGAGCGCUCAGGAAUCAU | SEQ ID NO: 188 |
| DRA-21 | GCUAUCAAAGGUAGGUGCUG | SEQ ID NO: 189 |
| DRA-22 | GUUACCUCUGGAGGUACUGG | SEQ ID NO: 190 |
| DRA-23 | UAGCACAGGGACUCCACUUA | SEQ ID NO: 191 |
| DRA-24 | UGAUGAAAAAUCCUAGCACA | SEQ ID NO: 192 |
| DRA-25 | UGAUGAGCGCUCAGGAAUCA | SEQ ID NO: 193 |
| DRA-27 | UUUGCCAGCUUUGAGGCUCA | SEQ ID NO: 194 |
| DRA-28 | AACUAUACUCCGAUCACCAA | SEQ ID NO: 195 |
| DRA-29 | AGAAGAACAUGUGAUCAUCC | SEQ ID NO: 196 |
| DRA-30 | AGCAGAGAGGGAGGUACCAU | SEQ ID NO: 197 |
| DRA-31 | AGCGCUUUGUCAUGAUUUCC | SEQ ID NO: 198 |
| DRA-32 | AGCUGUGGACAAAGCCAACC | SEQ ID NO: 199 |
| DRA-33 | AGGGAGGUACCAUUGGUGAU | SEQ ID NO: 200 |
| DRA-34 | AUAAACUCGCCUGAUUGGUC | SEQ ID NO: 201 |
| DRA-35 | AUUGGUGAUCGGAGUAUAGU | SEQ ID NO: 202 |
| DRA-36 | CCAUGUGGAUAUGGCAAAGA | SEQ ID NO: 203 |
| DRA-37 | CUUUGAGGCUCAAGGUGCAU | SEQ ID NO: 204 |
| DRA-38 | CUUUGUCAUGAUUUCCAGGU | SEQ ID NO: 205 |
| DRA-39 | GGAUAUGGCAAAGAAGGAGA | SEQ ID NO: 206 |
| DRA-40 | UAUCUGAAUCCUGACCAAUC | SEQ ID NO: 207 |
| DRA-41 | UGAGAUUUUCCAUGUGGAUA | SEQ ID NO: 208 |
| DRA-42 | UGAUCACAUGUUCUUCUGAA | SEQ ID NO: 209 |
| DRA-43 | UGCACCUUGAGCCUCAAAGC | SEQ ID NO: 210 |
| DRA-44 | UGCAUUGGCCAACAUAGCUG | SEQ ID NO: 211 |
| DRA-45 | UGGACGAUUUGCCAGCUUUG | SEQ ID NO: 212 |
| DRA-46 | UGGCAAAGAAGGAGACGGUC | SEQ ID NO: 213 |
| DRA-47 | UGGUGAUGAGAUUUUCCAUG | SEQ ID NO: 214 |
| DRA-48 | AAUGUCACGUGGCUUCGAAA | SEQ ID NO: 215 |
| DRA-49 | AGACAAGUUCACCCCACCAG | SEQ ID NO: 216 |
| DRA-50 | CAAUCCCUUGAUGAUGAAGA | SEQ ID NO: 217 |
| DRA-51 | GAACGCAGGGGGCCUCUGUA | SEQ ID NO: 218 |
| DRA-52 | CUGAGGACGUUUACGACUGC | SEQ ID NO: 219 |
| DRA-53 | GCGGAAAAGGUGGUCUUCCC | SEQ ID NO: 220 |
| DRA-54 | GGACGUUUACGACUGCAGGG | SEQ ID NO: 221 |
| DRA-55 | GUCGUAAACGUCCUCAGUUG | SEQ ID NO: 222 |
| DRA-56 | GUGAGCACAGUUACCUCUGG | SEQ ID NO: 223 |
| DRA-57 | GUGUCCCCCAGUACCUCCAG | SEQ ID NO: 224 |
| DRA-58 | UGAGGACGUUUACGACUGCA | SEQ ID NO: 225 |
| DRA-59 | AAUGGAAAACCUGUCACCAC | SEQ ID NO: 226 |
| DRA-60 | AGUGGAACUUGCGGAAAAGG | SEQ ID NO: 227 |
| DRA-61 | AUGAAACAGAUGAGGACGUU | SEQ ID NO: 228 |
| DRA-62 | CAGAGACAGUCUUCCUGCCC | SEQ ID NO: 229 |
| DRA-63 | CGUGACAUUGACCACUGGUG | SEQ ID NO: 230 |
| DRA-64 | UAUGAAACAGAUGAGGACGU | SEQ ID NO: 231 |
| DRA-65 | UCUGACACUCCUGUGGUGAC | SEQ ID NO: 232 |
| DRA-66 | AAACGUCCUCAGUUGAGGGC | SEQ ID NO: 233 |
| DRA-67 | UCGUAAACGUCCUCAGUUGA | SEQ ID NO: 234 |

### Example 1.2. Selection of gRNA Through Transfection into Raji Cell Line

7.5 μg of the obtained gRNA was incubated at 65° C. for 10 minutes to form a single strand. Then, 7.5 μg of Cas9 protein (Toolgen, TGEN_CP3 or Clontech, M0646T) was added thereto and incubation was performed at 25° C. for 10 minutes to prepare a Cas9-gRNA complex (RNP complex). The RNP complex was transfected into Raji cell line having 4×10⁵ cells with 4D-Nucleofector™ X Unit (Lonza, AAF-1002X) using SG Cell Line 4D-Nucleofector® X Kit S (Lonza, V4XC-3032). The transfected cells were incubated for 7 days, and then the expression level of HLA on the cell surface and the presence of a mutation in genomic DNA were identified.

### Example 1.3. Identification of Expression Level of HLA Using Flow Cytometer

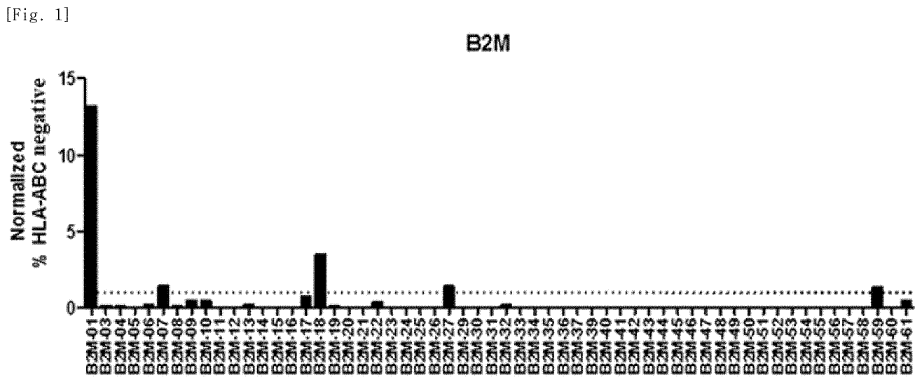

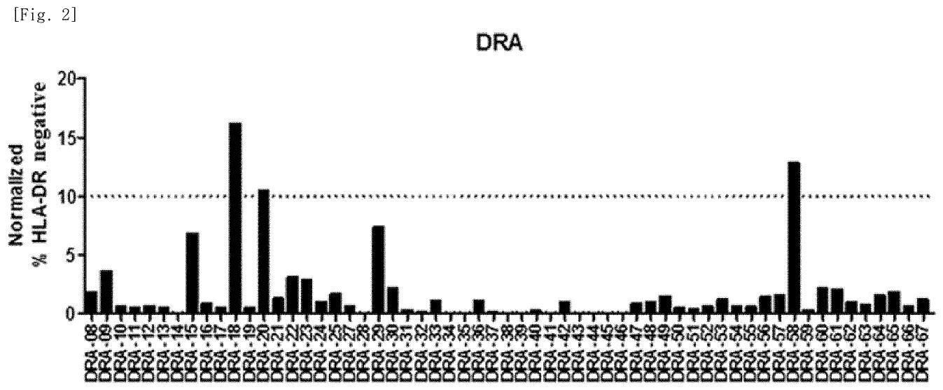

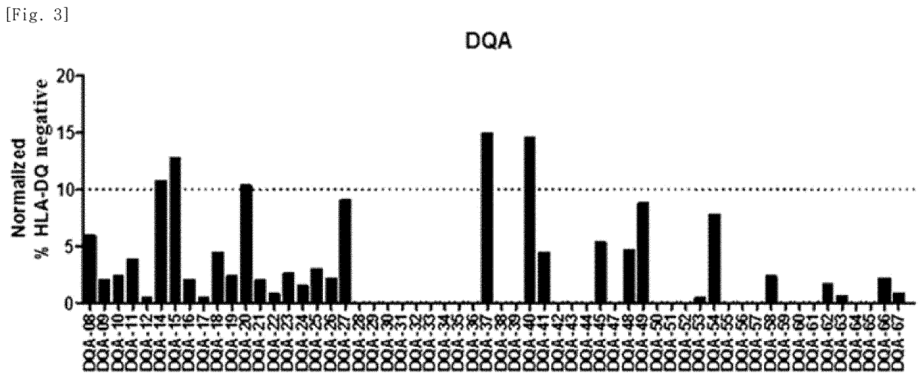

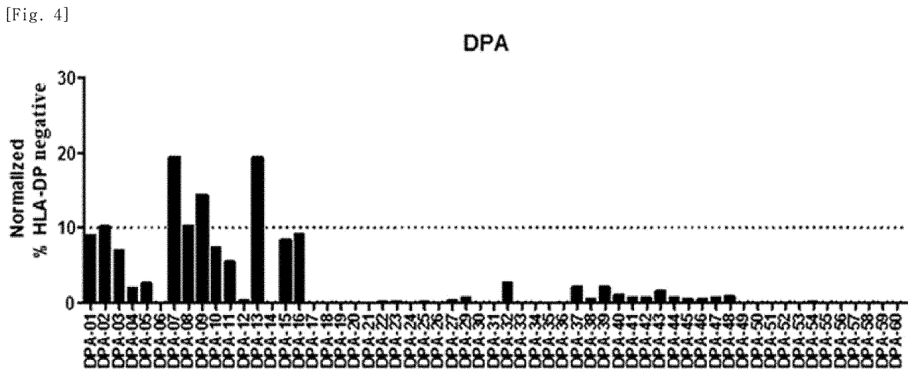

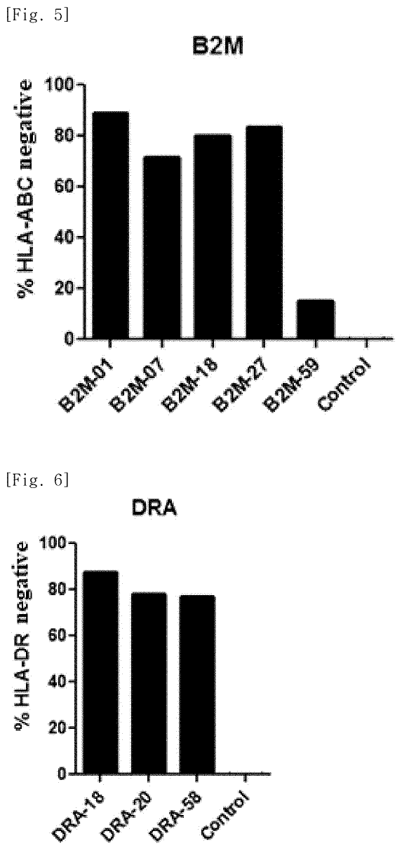

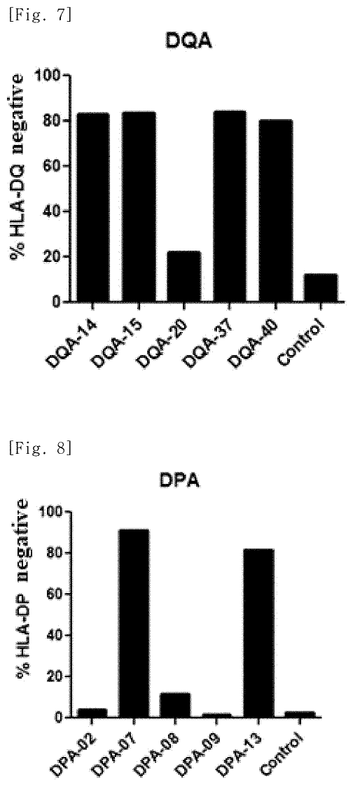

2×10⁵ cells of each of the RNP complex-transfected Raji cell line and control group Raji cell line were suspended in 100 μL of FACS buffer (1% FBS/sheath buffer) and prepared in a 5-mL tube. The cells were subjected to antibody treatment. Then, light was blocked for 30 minutes and incubation was performed at 4° C. As the antibodies, PE anti-HLA-ABC (Miltenyi Biotec, 130-101-448), PE anti-HLA-DR (Biolegend, 361605), PE anti-HLA-DQ (Biolegend, 318106), and PE anti-HLA-DP (Leinco Technologies, H130) were used. Thereafter, 3 mL of FACS buffer was added thereto and centrifugation was performed at 2,000 rpm for 3 minutes at 4° C. Then, the supernatant was removed to obtain a sample, and the sample was analyzed by LSR Fortessa. A total of 60 gRNAs were tested three times, each time using 20 gRNAs. The results for a value (normalized % HLA negative) calculated by subtracting the ‘% HLA negative’ value of the control group from the ‘% HLA negative’ value of each gRNA are illustrated in FIGS. 1 to 4. In addition, the results obtained by performing a re-experiment, at once, on the gRNAs having the value of 10 or higher (however, on the gRNAs having the value of 1 or higher in case of B2M-targeted gRNAs) are illustrated in FIGS. 5 to 8.

From the results in FIGS. 5 to 8, a total of 13 gRNAs capable of efficiently decreasing expression of each HLA were selected, of which 2 to 4 gRNAs were selected for respective targets (HLA-ABC, HLA-DQ, HLA-DP, and HLA-DR). Specifically, B2M-01, B2M-07, B2M-18, and B2M-27 gRNAs were selected for HLA-ABC; DQA-14, DQA-15, DQA-37, and DQA-40 were selected for HLA-DQ; DPA-07 and DPA-13 were selected for HLA-DP; and DRA-18, DRA-20, and DRA-58 were selected for HLA-DR.

### Example 1.4. Identification of Mutation in Genomic DNA of Target Gene

In order to identify whether the HLA-targeted gRNAs selected using flow cytometry cause a mutation in genomic DNA, the genomic DNA was analyzed using the Guide-it Mutation Detection Kit (Clontech, 631443) according to the manufacturer's instructions.

That is, 5×10⁵ cells of each of the RNP complex-transfected Raji cell line and the control group Raji cell line were centrifuged at 1,200 rpm for 5 minutes, and then the supernatant was removed. Then, 90 μL of extraction buffer 1 contained in the Guide-it Mutation Detection Kit (Clontech, 631443) was added thereto, and incubation was performed at 95° C. for 10 minutes. Then, 10 μL of extraction buffer 2 contained in the Guide-it Mutation Detection Kit (Clontech, 631443) was added thereto, and the DNA lysate obtained by pipetting was diluted in a ratio of 1:8 in pure water for PCR. PCR was performed with a PCR thermal cycler (FlexCycler2, Analytik Jena) using the diluted DNA lysate, and the selected gRNA and the analytical PCR primers for target genomic DNA as shown in Table below.

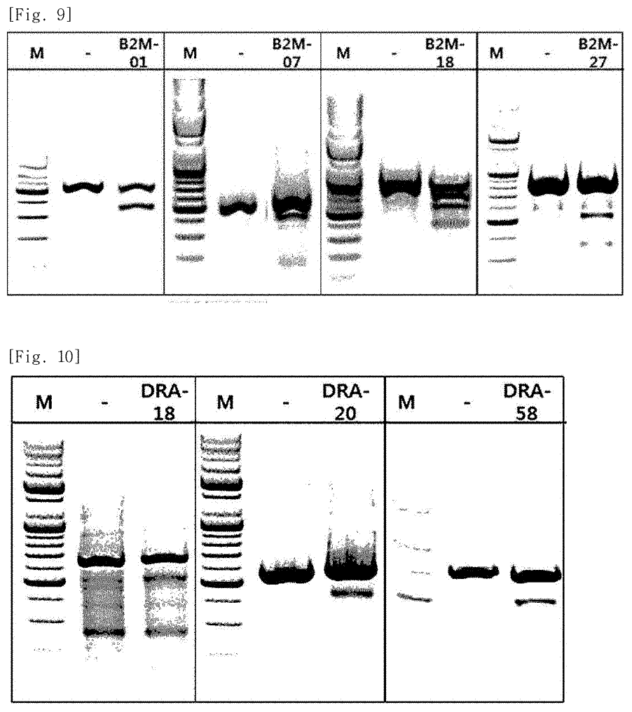

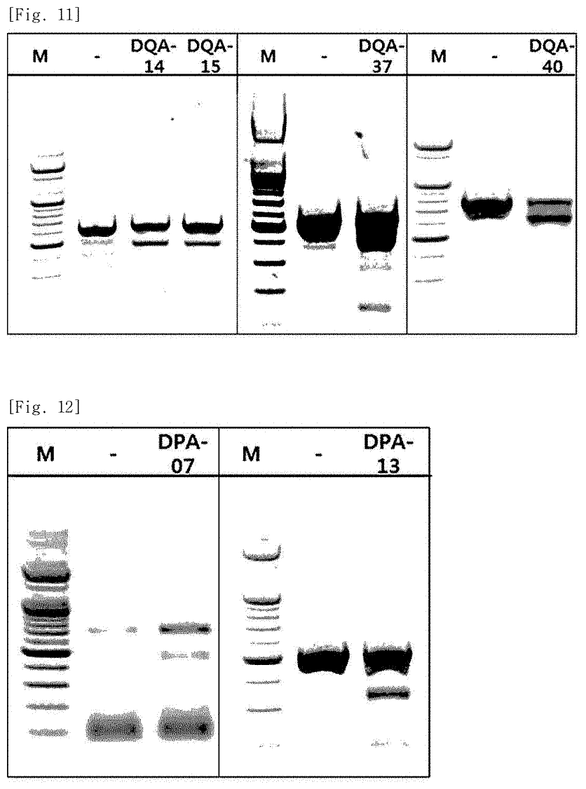

The following PCR parameters were used to produce the PCT product: pre-denaturation at 98° C. for 2 minutes, followed by 35 cycles of denaturation and annealing under a condition of at 98° C. for 10 seconds, at 60° C. for 15 seconds, and at 68° C. for 1 minute, followed by extension at 68° C. for 5 minutes. To denature and rehybridize the obtained PCR product, 5 μL of pure water for PCR was added to 10 μL of the PCR product. Subsequently, incubation was performed at 95° C. for 5 minutes, and then the temperature was changed under a condition where the temperature decreased by 2° C. per second from 95° C. to 85° C. and decreased by 0.1° C. per second from 85° C. to 25° C. Finally, 1 μL of Guide-it Resolvase was added thereto and incubation was performed at 37° C. for 30 minutes. Then, electrophoresis was performed on 1.5% agarose gel. The results are illustrated in FIGS. 9 to 12.

Guide-it resolvase-cleaved DNA fragments in the PCT product of the HLA-targeted gRNA-transfected cells were identified in the electrophoresis results. From these results, it was found that the 13 selected HLA-targeted gRNAs induced a mutation on their target genomic DNA.

TABLE 5

| | Estimated size of | | |
|---|---|---|---|
| gRNA name | cleaved DNA (bp) | PCR primer sequence | |
| B2M-1 | 173*308 | B2M E1-1F | CTGGCTTGGAGACAGGTGAC |
| | | | (SEQ ID NO: 242) |
| | | B2M E1-1R | GACGCTTATCGACGCCCTAA |
| | | | (SEQ ID NO: 243) |
| B2M-7 | 122*359 | B2M E1-1F | CTGGCTTGGAGACAGGTGAC |
| | | | (SEQ ID NO: 242) |
| | | B2M E1-1R | GACGCTTATCGACGCCCTAA |
| | | | (SEQ ID NO: 243) |
| B2M-18 | 326*474 | B2M E2-2F | CCCAAGTGAAATACCCTGGCA |
| | | | (SEQ ID NO: 244) |
| | | B2M E2-2R | AGCCCTTCCTACTAGCCTCA |
| | | | (SEQ ID NO: 245) |
| B2M-27 | 325*475 | B2M E2-2F | CCCAAGTGAAATACCCTGGCA |
| | | | (SEQ ID NO: 244) |
| | | B2M E2-2R | AGCCCTTCCTACTAGCCTCA |
| | | | (SEQ ID NO: 245) |
| DRA-18 | 475*131 | DRA E3-1F | AATTTCTTGGGGAGGGGGTG |
| | | | (SEQ ID NO: 246) |
| | | DRA E3-1R | AGCTGGATAGTAGGAGAAGACAGT |
| | | | (SEQ ID NO: 247) |
| DRA-20 | 382*141 | DRA E1-3F | GGGTTAAAGAGTCTGTCCGTGA |
| | | | (SEQ ID NO: 248) |
| | | DRA E1-3R | TGTCGAGACCACATAATACCTGT |
| | | | (SEQ ID NO: 249) |
| DRA-58 | 176*430 | DRA E3-1F | AATTTCTTGGGGAGGGGGTG |
| | | | (SEQ ID NO: 246) |
| | | DRA E3-1R | AGCTGGATAGTAGGAGAAGACAGT |
| | | | (SEQ ID NO: 247) |
| DQA-14 | 163*433 | DQA E1-1F | ACCTGACTTGGCAGGGTTTG |
| | | | (SEQ ID NO: 250) |
| | | DQA E1-1R | CCCAAGATCTACCACCGGAGA |
| | | | (SEQ ID NO: 251) |
| DQA-15 | 173*423 | DQA E1-1F | ACCTGACTTGGCAGGGTTTG |
| | | | (SEQ ID NO: 250) |
| | | DQA E1-1R | CCCAAGATCTACCACCGGAGA |
| | | | (SEQ ID NO: 251) |
| DQA-37 | 146*383 | DQA E3-2F | TGCTCCCAAGCAGAAGGTAA |
| | | | (SEQ ID NO: 252) |
| | | DQA E3-2R | AACCCATGAAGTGTGGAAAACAAG |
| | | | (SEQ ID NO: 253) |
| DQA-40 | 127*543 | DQA E3-1F | TCCCTCCATACCAGGGTTCA |
| | | | (SEQ ID NO: 254) |
| | | DQA E3-1R | AACTCATCCTTACCCCAGTGT |
| | | | (SEQ ID NO: 255) |
| DPA-7 | 206*401 | DPA 5F | TGTGTCAACTTATGCCGCGT |
| | | | (SEQ ID NO: 256) |
| | | DPA 5R | TTGGGAAACACGGTCACCTC |
| | | | (SEQ ID NO: 257) |
| DPA-13 | 178*322 | DPA E1-2F | TGTGAACTGGAGCTCTCTTGA |
| | | | (SEQ ID NO: 258) |
| | | DPA E1-2R | TATGAGGGCCAGAGGGAACAT |
| | | | (SEQ ID NO: 259) |

As such, the gRNAs capable of efficiently decreasing expression of respective HLAs through transfection in Raji cells were selected. In Examples 2 and 3, the selected gRNAs were used to prepare transformed NK cells, and then efficacy thereof was identified.

### Example 2. Preparation and Identification of HLA-I-Deleted Cells

### Example 2.1. Deletion of HLA-I in NK-92MI Cell Line

37.5 μg of B2M-01 gRNA was incubated at 65° C. for 10 minutes to form a single strand. Then, 37.5 g of Cas9 protein (Toolgen, TGEN_CP3) was added thereto and incubation was performed at 25° C. for 10 minutes to prepare a Cas9-gRNA complex (RNP complex). The RNP complex was transfected into NK-92MI cell line having 2×10⁶ cells with Nucleofector™ 2b (Lonza, AAB-1001) using Cell Line nucleofector Kit R (Lonza, VCA-1001). The transfected cells were incubated for 3 days, and then cell separation was performed using a cell separator.

### Example 2.2. Separation of HLA I Negative Cells

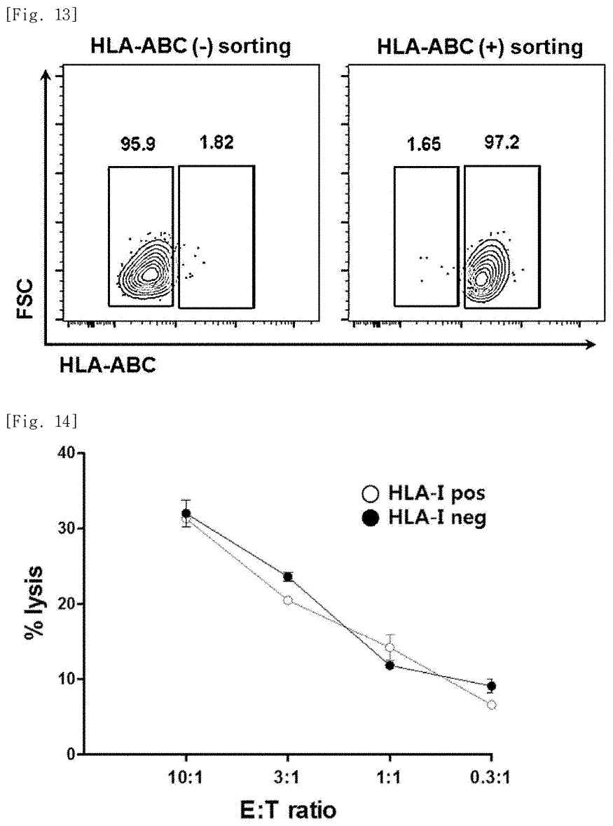

The B2M-01 RNP complex-transfected NK-92MI cell line was transferred to a 5-mL tube, and then treated with PE anti-HLA-ABC (Miltenyi Biotec, 130-101-448) and 7-AAD (Beckman Coulter, Inc., A07704). Then, light was blocked for 30 minutes and incubation was performed at 4° C. The stained cells were filtered using a filter top FACS tube (Falcon, 352235), and then HLA-I positive cells and HLA-I negative cells were separated using FACS Aria II (BD). The results are illustrated in FIG. 13. It was identified that the HLA-I negative cells have a purity of 95.9% and the HLA-I positive cells have a purity of 97.2%.

### Example 2.3. Evaluation of Cell-Killing Capacity of HLA-I-Deleted NK-92MI Cell Line

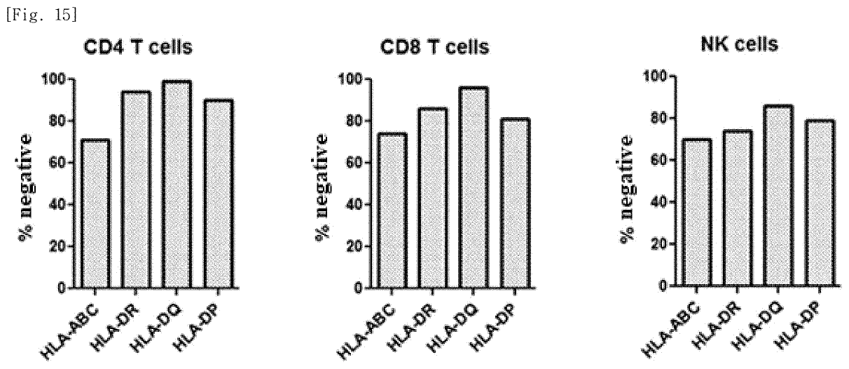

Using the HLA-I positive cells and the HLA-I negative cells, each of which had been incubated for 4 days after cell separation, cell-killing capacity thereof against K562 cell line was compared. The K562 cell line was stained with 30 μM Calcein-AM (Invitrogen, C3099) according to the manufacturer's instructions, and then incubated with the NK-92MI cell line on a U-bottom plate at an E:T ratio of 10:1, 3:1, 1:1, or 0.3:1. After 4 hours, each incubate was taken out by 100 μL, and the amount of Calcein-AM secreted by cell death was measured with a fluorometer (VictorTMX3, PerkinElmer). As a result, as illustrated in FIG. 14, it was identified that the HLA-I positive cells and the HLA-I negative cells had equivalent cell-killing capacity against the K562 cell line. From these results, it was found that deletion of HLA-I did not affect the cell-killing capacity.

### Example 3. Preparation and Identification of HLA-I- and HLA-T-Deleted Cells

### Example 3.1. Preparation and Incubation of NK Cells

Cryopreserved peripheral blood mononuclear cells (PBMCs) were rapidly dissolved in a water bath at 37° C., and then transferred to a 50-mL conical tube. With shaking of the tube, thawing media (RPMI, 11875-093+10% FBS+55 μM β-ME) was added dropwise thereto and mixed. Subsequently, centrifugation was performed at 1,200 rpm for 10 minutes at 4° C. to remove the supernatant, and resuspended in 10 mL of CellGro SCGM (CELLGENIX, 2001) media. Then, the number of cells was quantified. The cells were resuspended in culture media (CellGro SCGM+10 ng/mL OKT3+500 IU/mL IL-2+5% Human plasma) at a concentration of 1×10⁶ cells/mL, and then placed in Culture Bag (NIPRO, 87-352). Incubation was performed in a CO₂ incubator at 37° C. for 24 hours, and then transfection was performed.

### Example 3.2. Preparation of T Cells and Incubation Method

Cryopreserved peripheral blood mononuclear cells (PBMCs) were rapidly dissolved in a water bath at 37° C., and then transferred to a 50-mL conical tube. With shaking of the tube, thawing media (RPMI, 11875-093+10% FBS+55 μM β-ME) was added dropwise thereto and mixed. Subsequently, centrifugation was performed at 1,200 rpm for 10 minutes at 4° C. to remove the supernatant, and resuspended in 40 mL of MACS buffer (PBS+0.5% FBS+2 mM EDTA). Then, the number of cells was quantified. Treatment with 20 μL of CD3 microbeads (Miltenyi Biotec, 130-050-101) per 10⁷ cells was performed. Then, light was blocked for 15 minutes and incubated at 4° C. Subsequently, centrifugation was performed at 1,350 rpm for 8 minutes at 4° C. to remove the supernatant, and the resultant was resuspended in 500 μL of MACS buffer. Then, the resuspension was loaded onto an LS column (Miltenyi Biotec, 130-042-401) mounted on QuadroMACS separator (Miltenyi Biotec, 130-090-976). The LS column was washed 3 times with MACS buffer, and removed from the QuadroMACS separator. Then, the removed LS column was pressed with a plunger to obtain CD3 positive cells. The cells were resuspended at a concentration of 1×10⁶ cells/mL in T-cell culture media (X-VIV015 (Lonza, BE02-060Q)+40 μL/mL Dynabeads Human T-Activator CD3/CD28 (Gibco, 111.31D)+200 IU/mL IL-2+5% Human plasma) and then placed in Culture Bag (NIPRO, 87-352). Incubation was performed in a CO₂ incubator at 37° C. for 24 hours, and then transfection was performed.

### Example 3.3. Preparation of HLA-Deleted NK Cells and T Cells Using Selected gRNAs

37.5 μg of each of the gRNAs was incubated at 65° C. for 10 minutes to form a single strand. Then, 37.5 μg of Cas9 protein (Clontech, M0646T) was added thereto and incubation was performed at 25° C. for 10 minutes to prepare a Cas9-gRNA complex (RNP complex). In case of multiplex deletion, the sum of the amounts of respective gRNAs was set to 37.5 μg. The RNP complex was transfected into 2×10⁶ cells with 4D-Nucleofector™ X Unit (Lonza, AAF-1002X) using P3 Primary Cell 4D-Nucleofector® X Kit L (Lonza, V4XP-3024). The transfected cells were incubated for 3 days, and then production of cytokines was observed. The transfected cells were incubated for 14 days, and then it was identified, by flow cytometry, whether HLA expression was decreased.

### Example 3.4. Identification of Decreased Expression of HLA Using Flow Cytometry

For each of the RNP complex-transfected cells and the control group cells, 2×10⁵ cells were suspended in 100 μL of FACS buffer (1% FBS/sheath buffer) and prepared in a 5-mL tube. Cell staining was carried out over 3 times. For primary staining, anti-HLA-DP (Abcam, ab20897) was used; for secondary staining, PE Goat anti-mouse IgG (eBioscience, 12-4010-82) was used; and for tertiary staining, V450 anti-CD4 (BD, 560345), APC-Cy7 anti-CD8 (BD, 557834), BV510 anti-HLA-ABC (Biolegend, 311436), PE-Cy7 anti-HLA-DR (eBioscience, 25-9952-42), Alexa647 anti-HLA-DQ (BD, 564806) were used. On the other hand, in case of NK cells, BV421 anti-CD56 (Biolegend, 318328) was used in place of V450 anti-CD4. Each time, after the antibody treatment, light was blocked for 30 minutes and incubation was performed at 4° C. Thereafter, 3 mL of FACS buffer was added thereto, and centrifugation was performed at 2,000 rpm for 3 minutes at 4° C. to remove the supernatant. All stained samples were obtained and analyzed with LSR Fortessa. The results are illustrated in FIGS. 15 to 17.

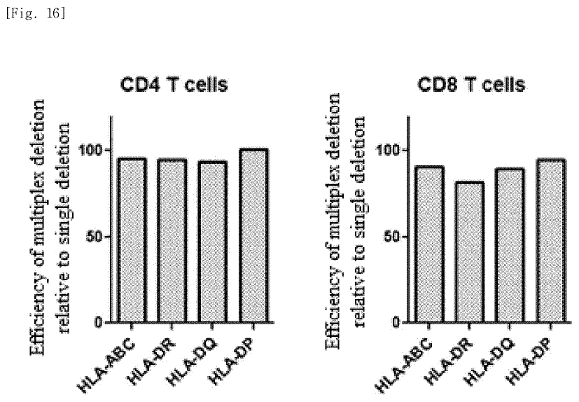

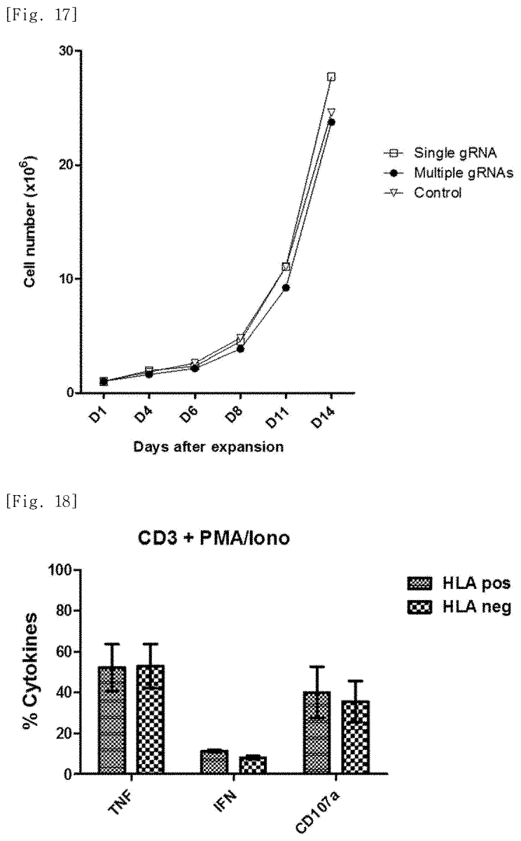

As gRNAs used to delete respective HLAs, B2M-01, DRA-20, DQA-14, and DPA-13 were used. From the results in FIG. 15, it was found that upon transfection with a single gRNA, deletion of the target HLA was achieved with high efficiency of at least 70% and up to 99%. From the results in FIG. 16, it was found that efficiency of the multiplex deletion was not remarkably decreased as compared with efficiency of the single deletion. When efficiency of the multiplex deletion was compared with respect to the single deletion, the efficiency was represented by multiplying a value, which was obtained by dividing the ‘% negative’ value of the single deletion by the ‘% negative’ value of the multiplex deletion, by 100. In addition, referring to the results in FIG. 17, a 14-day incubation rate for the RNP complex-transfected cells was found to be similar to that of the control group cells. In particular, it was identified that there was no difference in terms of incubation rate between single gRNA (DPA-13)-transfected cells and multiple gRNA-transfected cells.

### Example 3.5. Analysis of Activity of HLA-Deleted T Cells and NK Cells

For each of the RNP complex-transfected cells and the control cells, 1×10⁶ cells were subjected to treatment with PMA, ionomycin (Cell Stimulation Cocktail, eBioscience, 00-4970-03), and APC anti-CD107α (BD, 560664) followed by incubation, or were incubated with APC anti-CD107α and 2×10⁵ K562 cells. After 5 hours, treatment with PerCP-Cy5.5 anti-CD3 (Tonbo, 65-0038-T100), BV421 anti-CD56 (Biolegend, 318328), FITC anti-B2M (Biolegend, 316304), APC-Cy7 anti-HLA-ABC (Biolegend, 311426), and PE anti-HLA-DR/DP/DQ (Miltenyi Biotec, 130-104-827) was performed. Then, light was blocked for 30 minutes and incubation was performed at 4° C. so as to carry out surface staining.

Thereafter, 3 mL of FACS buffer was added thereto, and centrifugation was performed at 2,000 rpm for 3 minutes at 4° C. to remove the supernatant. Then, fixation and permeation were performed for 30 minutes using BD Cytofix/Cytoperm™ buffer (BD, 554722). Washing was performed twice with 1× Perm/Wash buffer (BD, 554723), and treatment with PE-Cy7 anti-TNF-α (eBioscience, 25-7349-82) and V500 anti-IFN-γ (BD, 554701) was performed. Light was blocked for 30 minutes and incubation was performed at 4° C. so as to carry out intracellular staining. Subsequently, 3 mL of FACS buffer was added thereto, and centrifugation was performed at 2,000 rpm for 3 minutes at 4° C. to remove the supernatant. Then, washing was performed twice with IX Perm/Wash buffer, and cytokine production in T cells and NK cells was analyzed with flow cytometry. The results are illustrated in FIGS. 18 and 19.

In FIG. 18, it was identified that even when HLA was deleted, the amounts of TNF-α, IFN-γ, and CD107a, which are secreted when T cells were activated, are not different from HLA positive cells. Also in FIG. 19, it was identified that even when HLA was deleted, the amounts of TNF-α, IFN-γ, and CD107a, which are secreted when NK cells are activated, were not different from HLA positive cells. From these results, it was found that activity of NK cells was maintained even when HLA-I and HLA-II were deleted.

### Example 4. Synthesis of HLA-E-Expressing Vector and Identification of Expression

### Example 4.1. Evaluation of Cell-Killing Capacity of NK Cells Against HLA-I-Deleted Raji Cell Line

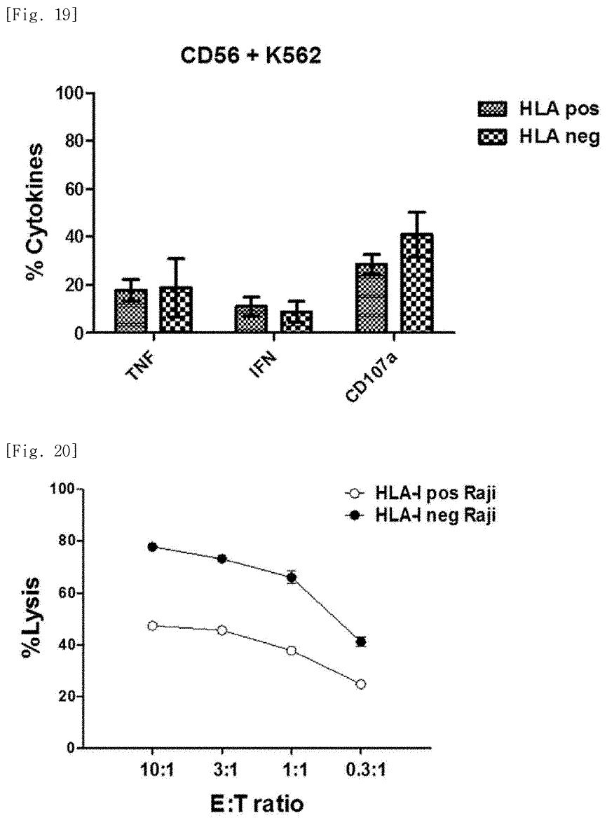

In order to identify whether cell-killing capacity of NK cells is increased in HLA-I-deleted cells, Raji cell line was transfected with B2M-01 RNP complex, and HLA-I positive cells and HLA-I negative cells were separated using a cell separator. The respective cells were stained with Calcein-AM according to the manufacturer's instructions, and then 1×10⁴ cells were incubated with NK-92MI cell line on a U-bottom plate at an E:T ratio of 10:1, 3:1, 1:1, or 0.3:1. After 5 hours, the amount of Calcein-AM secreted by cell death was measured with a fluorometer. As illustrated in FIG. 20, it was identified that cell-killing capacity of NK cells was increased in HLA-I negative cells as compared with HLA-I positive cells.

### Example 4.2. Synthesis of HLA-E Vector

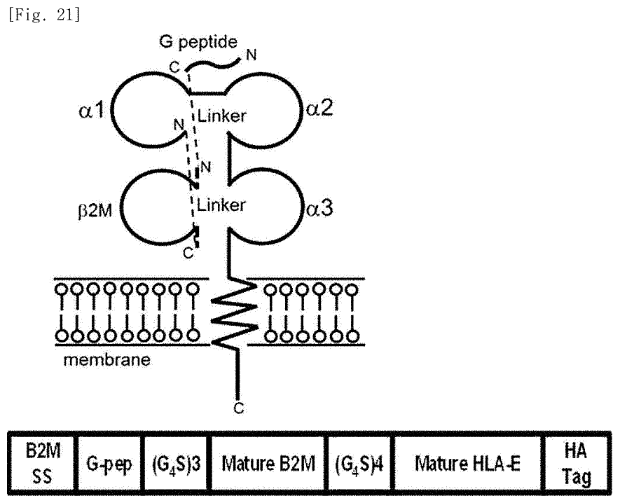

In order to avoid a cell-killing phenomenon caused by NK cells which occurs when cells are transfected with B2M RNP complex so as to delete HLA-I, as in Example 4.1, an HLA-E vector for introducing HLA-E into the cells was synthesized. That is, transformed HLA-E (G-B2M-HLA-E) was synthesized in which B2M (SEQ ID NO: 237) was linked, via three first G₄S linkers (SEQ ID NO: 238), to G-peptide (SEQ ID NO: 236) connected to B2M signal peptide (B2M SS, SEQ ID NO: 235), and B2M was linked, via four second G₄S linkers (SEQ ID NO: 241), to HLA-E (SEQ ID NO: 240) attached with HA tag (SEQ ID NO: 239). The respective sequences are shown in Table 6 below. The synthesized transformed HLA-E was cloned by insertion into the pLVX-EF1α-IRES-Puro Vector (Clontech, 631988), and the structure thereof is as illustrated in FIG. 21.

TABLE 6

| Transformed HLA-E | Amino acid sequence |
|---|---|
| B2M SS | MSRSVALAVLALLSLSGLEA (SEQ ID NO: 235) |
| G-peptide | VMAPRTLFL (SEQ ID NO: 236) |
| B2M | IQRTPKIQVYSRHPAENGKSNFLNCYVSGFHPSDIEVDLLKNGE |
| | RIEKVEHSDLSFSKDWSFYLLYYTEFTPTEKDEYACRVNHVTL |
| | SQPKIVKWDRDM (SEQ ID NO: 237) |
| First linker | GGGGSGGGGSGGGGS (SEQ ID NO: 238) |
| (G₄S linker 1) | |
| HA tag | YPYDVPDYA (SEQ ID NO: 239) |
| HLA-E | GSHSLKYFHTSVSRPGRGEPRFISVGYVDDTQFVRFDNDAASP |
| | RMVPRAPWMEQEGSEYWDRETRSARDTAQIFRVNLRTLRGY |
| | YNQSEAGSHTLQWMHGCELGPDGRFLRGYEQFAYDGKDYLT |
| | LNEDLRSWTAVDTAAQISEQKSNDASEAEHQRAYLEDTCVEW |
| | LHKYLEKGKETLLHLEPPKTHVTHHPISDHEATLRCWALGFYP |
| | AEITLTWQQDGEGHTQDTELVETRPAGDGTFQKWAAVVVPSG |
| | EEQRYTCHVQHEGLPEPVTLRWKPASQPTIPIVGIIAGLVLLGS |
| | VVSGAVVAAVIWRKKSSGGKGGSYSKAEWSDSAQGSESHSL |
| | (SEQ ID NO: 240) |
| Second linker | GGGGSGGGGSGGGGSGGGGS (SEQ ID NO: 241) |
| (G₄S linker 2) | |

### Example 4.3. Expression of HLA-E Through Transduction into K562 Cell Line and Identification Thereof

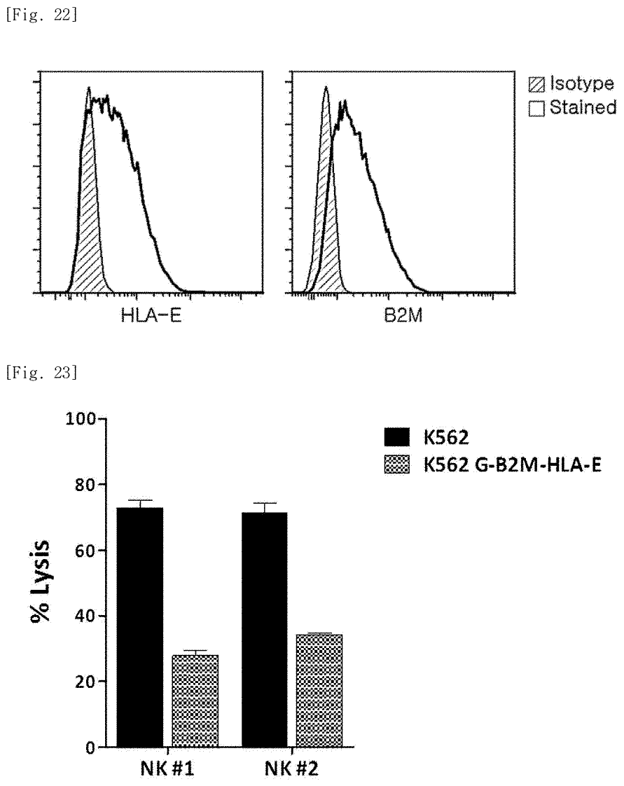

The transformed HLA-E-inserted pLVX-EF1α-IRES-Puro Vector was transfected into 293T cell line together with a lenti viral packaging vector. After 3 days, the lentiviral supernatant was obtained through a 0.45-μm filter. K562 cell line was subjected to treatment with the lentiviral supernatant, and centrifugation was performed at 3,000 rpm for 1 hour at 32° C. After 3 days, 1×10⁶ cells were transferred to a 5-mL tube, and cell surface staining was carried out for 30 minutes with PE-Cy7 anti-HLA-E (Biolegend, 342608) and APC anti-B2M (Biolegend, 316312). Washing with FACS buffer was performed, and then expression of HLA-E and B2M was checked with flow cytometry. As can be seen from FIG. 22, it was identified that HLA-E and B2M were expressed at high levels in the K562 cell line expressing the transformed HLA-E.

### Example 4.4. Evaluation of Cell-Killing Capacity in HLA-E-Introduced Cells

Each of the K562 cell line expressing the transformed HLA-E and the control group K562 cell line was stained with Calcein-AM according to the manufacturer's instructions, and then 1×10⁴ cells were incubated with NK cells on a U-bottom plate at an E:T ratio of 10:1, 3:1, 1:1, or 0.3:1. After 5 hours, 100 μL was taken out from each incubate, and the amount of Calcein-AM secreted by cell death was measured with a fluorometer (VictorTMX3, PerkinElmer).

As can be seen from FIG. 23, it was identified that a cell death phenomenon caused by NK cells was significantly decreased in case of the K562 cell line (K562 G-B2M-HLA-E) expressing the transformed HLA-E as compared with the control group K562 cell line (K562). From these results, it was found that expression of HLA-E could prevent cell death caused by NK cells.

### Example 5. Introduction of HLA-E to HLA-I- and HLA-II-Deleted NK Cells

HLA-E to which G-peptide was bound was introduced to the HLA-I- and HLA-II-deleted NK cells prepared in Example 3 using the HLA-E vector prepared as in Example 4.2, to prepare transformed NK cells.

1. A guide RNA molecule that is complementary to a nucleic acid encoding β2-microglobulin (B2M), the guide RNA molecule comprising any one nucleic acid sequence selected from the group consisting of SEQ ID NO: 1, SEQ ID NO: 6, SEQ ID NO: 17, and SEQ ID NO: 26.

2. A guide RNA molecule that is complementary to a nucleic acid encoding HLA-DQ, the guide RNA molecule comprising any one nucleic acid sequence selected from the group consisting of SEQ ID NO: 64, SEQ ID NO: 65, SEQ ID NO: 87, and SEQ ID NO: 90.

3. A guide RNA molecule that is complementary to a nucleic acid encoding HLA-DP, the guide RNA molecule comprising the nucleic acid sequence of SEQ ID NO: 123 or SEQ ID NO: 129.

4. A guide RNA molecule that is complementary to a nucleic acid encoding HLA-DR, the guide RNA molecule comprising any one nucleic acid sequence selected from the group consisting of SEQ ID NO: 186, SEQ ID NO: 188, and SEQ ID NO: 225.

5. A composition comprising as active ingredients:
the guide RNA molecule of claim 1 or a nucleic acid encoding the guide RNA molecule; and
an RNA-guided endonuclease or a nucleic acid encoding the RNA-guided endonuclease.

6. The composition of claim 5, wherein the RNA-guided endonuclease is any one selected from the group consisting of Cas1, Cas1B, Cas2, Cas3, Cas4, Cas5, Cas6, Cas7, Cas8, Cas9, Cas10, Cas12a, Cas12b, Cas12c, Cas12d, Cas12e, Cas 13a, Cas 13b, Cas 13c, Cas 13d, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

7. A transformed cell in which expression of MHC I cell membrane receptor and MHC II cell membrane receptor is inhibited.

8. The transformed cell of claim 7, wherein the transformed cell expresses a peptide antigen on the cell membrane surface.

9. The transformed cell of claim 8, wherein the peptide antigen is G-peptide.

10. The transformed cell of claim 9, wherein the G-peptide is bound to a modified MHC I cell membrane receptor.

11. The transformed cell of claim 10, wherein the modified MHC I cell membrane receptor has a structure in which HLA-E and B2M are linked.

12. The transformed cell of claim 11, wherein the C-terminus of the B2M is linked, via a first linker, to the N-terminus of al of the HLA-E, and the C-terminus of the G-peptide is linked, via a second linker, to the N-terminus of the B2M in the modified MHC I cell membrane receptor.

13. The transformed cell of claim 12, wherein the G-peptide has the sequence of SEQ ID NO: 236.

14. The transformed cell of claim 12, wherein the HLA-E has the sequence of SEQ ID NO: 240.

15. The transformed cell of claim 12, wherein the B2M has the sequence of SEQ ID NO: 237.

16. The transformed cell of claim 12, wherein the first linker has the sequence of SEQ ID NO: 238.

17. The transformed cell of claim 12, wherein the second linker has the sequence of SEQ ID NO: 241.

18. The transformed cell of claim 7, wherein modification in a gene encoding the MHC I cell membrane receptor is performed using the guide RNA molecule of claim 1.

19. The transformed cell of claim 7, wherein modification in DQ, DP, and DR genes encoding the MHC II cell membrane receptor is performed using
a guide RNA molecule that is complementary to a nucleic acid encoding HLA-DQ, the guide RNA molecule comprising any one nucleic acid sequence selected from the group consisting of SEQ ID NO: 64, SEQ ID NO: 65, SEQ ID NO: 87, and SEQ ID NO: 90,
a guide RNA molecule that is complementary to a nucleic acid encoding HLA-DP, the guide RNA molecule comprising the nucleic acid sequence of SEQ ID NO: 123 or SEQ ID NO: 129; and
a guide RNA molecule that is complementary to a nucleic acid encoding HLA-DR, the guide RNA molecule comprising any one nucleic acid sequence selected from the group consisting of SEQ ID NO: 186, SEQ ID NO: 188, and SEQ ID NO: 225, respectively.

20. The transformed cell of claim 7, wherein the transformed cell is a therapeutic allogeneic cell.

21. The transformed cell of claim 20, wherein the therapeutic allogeneic cell is an immune cell or stem cell.

22. The transformed cell of claim 21, wherein the immune cell is an NK cell or T cell.

23. A pharmaceutical composition comprising as an active ingredient the transformed cell of claim 7.

24. The method of claim 26, wherein the cancer is any one selected from the group consisting of chronic lymphocytic leukemia (CLL), B-cell acute lymphocytic leukemia (B-ALL), acute lymphoblastic leukemia, acute myeloid leukemia, lymphoma, non-Hodgkin's lymphoma (NHL), multiple myeloma, blood cancer, gastric cancer, liver cancer, pancreatic cancer, colorectal cancer, lung cancer, breast cancer, ovarian cancer, skin cancer, melanoma, sarcoma, prostate cancer, esophageal cancer, hepatocellular carcinoma, astrocytoma, mesothelioma, head and neck cancer, and medulloblastoma.

25. The method of claim 26, wherein the infectious disease is any one selected from the group consisting of hepatitis B, hepatitis C, human papilloma virus (HPV) infection, cytomegalovirus infection, Epstein Barr virus (EBV) infection, viral respiratory disease, and influenza.

26. A method for treating cancer, an infectious disease, a degenerative disease, a hereditary disease, or an immune disease, comprising administering to a subject, the pharmaceutical composition of claim 23.

27. The method of claim 26, wherein the administration is performed via any one route selected from the group consisting of intravenous, intramuscular, intradermal, subcutaneous, intraperitoneal, intraarteriolar, intraventricular, intralesional, intrathecal, topical, and a combination thereof.

28. (canceled)
